(19) United States
(12) Patent Application Publication
(10) Pub. No.: US 2011/0208261 A1
(43) Pub. Date: Aug. 25, 2011

(54) SYSTEMS AND METHODS FOR ASSESSING AND REPROGRAMMING SENSING VECTORS FOR USE WITH AN IMPLANTABLE CARDIAC RHYTHM MANAGEMENT DEVICE

(75) Inventors: Paul A. Levine, Santa Clarita, CA (US); Eliot L. Ostrow, Sunnyvale, CA (US)

(73) Assignee: PACESETTER, INC., Sylmar, CA (US)

(21) Appl. No.: 12/712,037

(22) Filed: Feb. 24, 2010

(51) Int. Cl. A61N 1/08

(52) U.S. Cl. 607/27

(57) ABSTRACT

Techniques are provided for use with a pacemaker or other implantable medical device capable of sensing electrical signals along a set of programmable sensing vectors. In one example, electrical cardiac signals are sensed within a patient using a primary sensing vector connected to a primary sensing channel for use in controlling the delivery of therapy. If the device detects a significant drop in key signal parameters such as peak signal amplitude or slew rate, an assessment is made whether an alternate sensing vector provides improved cardiac signal sensing. During the assessment, the device can continue to sense signals along the primary channel for the purposes of controlling therapy while alternate vectors are assessed in the background. If it is determined that an alternate sensing vector provides improved cardiac signal sensing, the primary sensing channel can be switched to the alternate sensing vector for use in controlling further therapy.

### FIELD OF THE INVENTION

The invention relates generally to implantable cardiac rhythm management devices such as pacemakers and implantable cardioverter defibrillators (ICDs) and in particular to techniques for reprogramming lead sensing configurations for use in efficiently sensing electrical cardiac signals.

### BACKGROUND OF THE INVENTION

An implantable cardiac rhythm management device is a type of implantable medical device (IMD) that delivers therapy to the heart of a patient in which the device is implanted. For example, a pacemaker recognizes various cardiac arrhythmias and delivers electrical pacing pulses to the heart in an effort to remedy the arrhythmias. An implantable cardioverter/defibrillator (ICD) additionally or alternatively recognizes ventricular tachycardia (VT) and ventricular fibrillation (VF) and delivers electrical shocks or other therapies to terminate these ventricular tachyarrhythmias. Pacemakers, ICDs and other cardiac rhythm management devices typically use a set of leads implanted on or in the heart to sense electrical cardiac signals. Each lead may include a set of electrodes that can be electrically connected to the device to sense cardiac signals within the heart along various sensing vectors.

Current state-of-the-art devices permit cardiac signals to be sensed using several possible electrode configurations. For example, one electrical cardiac signal might be derived from voltage signals sensed along a vector between the right ventricular tip (RV) tip electrode and the RVring electrode; whereas another signal might be derived from voltage signals sensed along a vector between the right atrial (RA) tip electrode and the housing or “can” of the device itself. Typically, cardiac signals that are sensed between two electrodes. The electrodes may be located either in or on the heart (or one or more could be located outside the heart although still within the body.) If both electrodes are located within the heart and usually on the same lead within one or two centimeters of each other, this is termed bipolar. When only a single electrode is located within the heart and the other is outside the heart such as the device housing, this is termed unipolar. The phrasing unipolar has also, in recent years, sometimes been used to describe an electrode dipole when both electrodes, although physically located in the heart, are widely separated such as an LVtip electrode and the RVring or RVcoil electrode; alternatively, this configuration has sometimes been referred to as “widely spaced bipolar.” Technically, however for sensing to occur, there must be two electrodes. Sensing cannot occur with only a single electrode. The implanted device typically analyzes the cardiac signals occurring between an electrode pair to detect various events, such as atrial depolarization events (P-waves), ventricular depolarization events (R-waves), and ventricular repolarization events (T-waves). Strictly speaking, P-waves, R-waves and T-waves are features of a surface electrocardiogram (ECG). For generality and convenience, the terms P-waves, R-waves and T-waves are used herein to refer to their internal counterparts, which represent features within intracardiac electrograms (IEGMs.)

At the time of implantation, an effort is made to find a position for the lead in each heart chamber that provides a low capture threshold and a large signal for sensing purposes. If the signal is poor, the lead is repositioned until an adequate signal is identified and the lead is then secured in that location. However, during a period of time following implant (ranging from minutes to days to months), the signal amplitude might decrease to extremely low levels compromising the ability to properly sense electrical cardiac signals. If the implanted device is an ICD or current generation low voltage devices with automatic sensing adjustment, the very low signal amplitude means that the pulse generator needs to be very sensitive in order to detect these very small signals. A very sensitive system also increases the likelihood of detecting physiologically inappropriate signals such as T waves as well as extra-cardiac signals such as environmental electromagnetic signals (EMI). The sensitivity of the pulse generator is described as the smallest signal (in terms of millivolts—mV) that can be detected by the pulse generator after being processed by the sensing circuit with its filters and amplifiers. Hence, if one is to program a “high” sensitivity meaning that it is capable of detecting a very small signal, the sensitivity will be programmed to a low mV value. A “low” sensitivity meaning that the pulse generator can only detect a very large signal, smaller signals will be ignored and the pulse generator will be set to a high mV value. By way of illustration, a 0.5 mV sensitivity is more sensitive (described as a higher sensitivity) than a 2.0 mV sensitivity. Any detected signal, in this case, on the ventricular channel will be labeled an R-wave and, if the interval between successive R-waves is sufficiently short (i.e. the rate is fast), the rhythm will be labeled as VT or VF and antitachyarrhythmia therapy could be delivered depending on the programmed detection parameters. If the rhythm was normal but the pulse generator was seeing both the native QRS and the T wave, the rate would be labeled as being fast and the antitachyarrhythmia therapy would be delivered even though it was physiologically inappropriate. T-wave “oversensing” can result in many inappropriate shocks. The shocks can be painful, frightening to the patient and family, compromising to the quality of life of the patient and wasteful of energy in the power supply of the ICD, potentially shortening its longevity significantly. The opposite problem, “undersensing” can also occur, wherein cardiac events such as R-waves are too small to be properly detected. If so, abnormal rhythm might not be not recognized (such as VF or atrial fibrillation (AF)) precluding delivery of appropriate therapy.

There can be many causes for the decrease in amplitude of the signal including, but not limited to, lead migration or dislodgment such that it is no longer at the original location, alteration in orientation of the lead position such that the dipole is improper for the intrinsic wave front, progression of disease in the patient, metabolic abnormalities, side-effects of pharmacologic agents, and other factors. If the problem is identified during the implant and before the implantation pocket is closed, the physician can reposition the lead, although this is an imposition as it extends the length of the procedure, which increases the likelihood of post-operative complications such as infection and is generally frustrating to the clinician. If the decrease in signal amplitude occurs later or is only recognized after the pocket is closed, there are two primary options, both of which are generally limited. One is to take the patient back to the operating room, open the pocket, free the lead from the surrounding fibrous tissue and then reposition or replace the lead in another location. The other option is to manage the problem by noninvasively increasing the sensitivity and/or by making other adjustments to the sensing algorithms of the device (e.g., decay delay, etc.). This is not always successful and it requires a visit to the clinic or physician's office.

State-of-the-art pulse generators ICD, pacemaker and CRT] systems are being designed that have more than one lead in the ventricle and many such leads are likely to have multiple electrodes. (An example of a lead that can include sixteen (or even more) electrodes is disclosed in U.S. Patent Publication No. 2006/0058588 of Zdeblick.) These multi-electrode leads provide a variety of potential sensing vectors from which a clinician can identify a particular vector that significantly increases the signal amplitude, thus providing a means for managing the problems associated with the decrease in signal amplitude, oversensing, or undersensing associated with the originally selected electrode pair for sensing. Heretofore, however, there has been no convenient system for analyzing the many possible sensing vectors to identify an optimal sensing vector or for allowing the device itself to make such assessments and/or adjustments. (In this regard, there are some systems that can monitor and automatically adjust the device's sensitivity, and others that allow reprogramming between dedicated bipolar (tip-ring) and integrated bipolar (tip-coil) configurations; there are none that the applicants are aware of that automatically change the sensing configuration based on the quality of the sensed signal.)

As an example, when Pacesetter Inc. (the Assignee of rights to the present application) incorporated pace and sense polarity programmability in its Paragon model 2010 dual chamber pacemaker introduced in 1985-1986, the atrial and ventricular channels could be programmed to one of three different configurations for sensing: 1) standard Tip-Ring bipolar; 2) standard Tip-Case unipolar; or 3) a special unipolar configuration from the Ring to the Case. Further, while the system was programmed to a bipolar configuration, one could both examine the intracardiac electrograms associated with the other two configurations. At present time, Pacesetter programming systems can configure the sensing configuration for an assessment of the sensing threshold that is different from the programmed sensitivity, giving the clinician increased control while reducing risk because he or she can evaluate all the different configurations without having to first permanently program the configuration in order to test it. Hence, if the pacemaker is programmed to the bipolar sensing configuration and the bipolar signal is poor, the clinician can examine the telemetered electrogram and assess the sensing threshold (R-wave or P-wave amplitude) in the other two unipolar configurations that are available. Then the physician can make a decision as to which configuration is best for the patient and program that configuration.

However, there is considerable room for further improvement. A primary challenge is to effectively manage a patient who, for one reason or another, has experienced a marked decrease in signal amplitude from either the atrium or the ventricle with its attendant problems of oversensing of non-physiologic and physiologically inappropriate signals when the sensitivity setting of the device is increased or failing to sense appropriate physiologic signals resulting in competition, which can compromise hemodynamics and may induce tachyarrhythmias at the current programmed sensitivity setting.

Accordingly, it would be desirable to provide improved systems and techniques for meeting this challenge. It is to this end that the present invention is primarily directed.

### SUMMARY OF THE INVENTION

In an exemplary embodiment, a method is provided for use with an implantable medical device capable of sensing electrical cardiac signals along a plurality of sensing channels connected to a selectable sensing vector of a lead system. The lead “system” is comprised of all the implanted leads that are connected to the implantable medical device (which may also be referred to as a pulse generator, at least in implementations where the device is equipped to deliver some form of electrical stimulation pulses) although sensing itself only occurs between specific electrode pairs of the leads. The implantable medical device need not be restricted to the cardiac system but is applicable to any stimulating system in the body where detection of intrinsic electrical activity is integral to the performance of the system. Herein, implantable medical devices for detecting cardiac signals are primarily described as there is the greatest body of experience with such devices, but these are merely illustrative examples.

In the exemplary embodiment, electrical signals are sensed within the patient using a primary sensing vector connected to a primary sensing channel for use in controlling the delivery of therapy. Additional electrical signals are concurrently sensed within the patient using an alternate sensing vector connected to an alternate sensing channel. While continuing to sense signals using the primary sensing vector, an assessment is made as to whether the alternate sensing vector provides improved sensing over the primary sensing vector. If so, the primary sensing channel is switched from the primary sensing vector to the alternate sensing vector for sensing further electrical signals for use in controlling further therapy. That is, the alternate sensing channel allows for “background” processing of cardiac signals along alternate sensing vectors so that the primary sensing channel is not affected. This allows for delivery of therapy during the assessment procedure. Moreover, at least in examples where the device itself is capable of performing the assessment on its own, this feature allows the device to continuously or periodically monitor for other sensing vectors that might provide improved sensing as compared to the currently-programmed primary sensing vector.

Depending on the particular implementation, the assessment procedure can be triggered in response the detection of a significant change in a selected parameter of the electrical signals sensed along the primary sensing vector—such as a declining trend in peak signal amplitude or a declining trend in slew rate. That is, the steps of sensing additional electrical signals within the patient using an alternate sensing vector and assessing whether the alternate sensing vector provides improved sensing over the primary sensing vector can be performed in response to the detection of the significant change in the selected parameter. Herein, the term trend refers to generally long-term changes in signal parameters, i.e. changes occurring over at least several cardiac cycles rather than during a single cardiac cycle. Typically, trends occurring over days or weeks are detected. Moreover, note that the term “signal amplitude” can be broadly representative of various amplitude factors, such as slew rate, degree of fractionation and other facets of sensing.

In an illustrative example, wherein the electrical signals are cardiac signals, a triggering threshold is established (as either a default value or specifically programmed by the clinician.) If the device detects a drop in cardiac signal amplitude (or magnitude) below the threshold, the assessment is then made whether an alternate sensing vector provides improved cardiac signal sensing over the primary sensing vector and, if so, the primary sensing channel is selectively switched from the current sensing vector to the alternate sensing vector for sensing further cardiac signals for use in controlling further therapy. In one particular example, the device itself automatically switches the sensing vectors (assuming this feature has been enabled by the clinician in advance.) In another example, the results of the systematic evaluation are tabulated and presented to the physician at a follow-up or remote evaluation and the sensing vector is then reprogrammed by an external programmer device under clinician supervision. Depending upon the lead system, these techniques may be applied to, e.g., RV leads, LV leads, atrial leads, or any combination thereof as well as systems capable of sensing and stimulating in other organ systems.

Although summarized with respect to an example having a primary sensing vector connected a primary sensing channel, it should be understood that the technique can be applied to devices having an arbitrary number of sensing channels derived from an arbitrary number of sensing vectors. That is, the device can detect a drop in signal amplitude on any pair of electrodes currently in use and then (in some examples) can automatically reprogram its operation to use an alternate pair of electrodes resulting in a change in sensing vector. In other examples, the selection of alternative sensing vectors is instead performed by an external programming device under clinician supervision. That is, various alternative “candidate” sensing configurations can be displayed such that the clinician can then select a particular sensing configuration for use in reprogramming the device.

In one example, the device is enabled by the clinician to automatically control the reprogramming of its sensing vectors. If the signal amplitude (sensed signal) falls below a predefined or programmable threshold value, it initiates a search of other electrode pairs to assess signal amplitude. The device then automatically selects the largest signal amplitude and auto-programs that value.

In another example, the assessment process is a programmer controlled evaluation where, when instructed to do so (based on commands delivered via the external programmer), the device systematically examines all other sensing configurations available to the device by sequentially connecting the other sensing vectors to an alternate sensing channel and examining the strength of the cardiac signals sensed thereby. In this manner, the programmer can report the signal amplitudes for these alternate sensing vectors that allow the clinician to select and enable an improved sensing vector (electrode pair) over the currently programmed sensing vector.

In an automatic implementation, the implantable device can monitor the signal amplitude for the programmed sensing configuration. If that signal amplitude falls below a pre-defined (nominal) value or a physician programmed value, the device automatically evaluates the signal amplitude (or other features of the signal) using different electrode pairs. The device then identifies the best of the alternative sensing vectors and automatically switches the primary sensing channel to that vector for continued sensing or stores the measurements for reporting to the clinician during a subsequent office evaluation (monitor mode) or automatically. In one example, the “best” sensing vector is the vector providing the largest signal amplitude. In other examples, other criteria can be used to choose among alternate vectors. For example, if multiple vectors are deemed better than the primary vector, the system may choose the vector with the closest bipole among various candidate vectors, even if that vector does not have the largest signal.

In another implementation, the implantable device is programmed to a monitor mode to periodically assess the signal amplitude and provide a summary of the measurements between various electrodes on data that is retrieved via the programmer at a clinic evaluation or remotely. The physician can then select one of the electrode pairs for sensing and program this selection via the programmer.

A silent alarm can be generated to notify a clinician of the programming change and suitable diagnostics are recorded for subsequent clinician review that identifies the change in the detected reduction in signal amplitude (indicative of a potential sensing problem) and the corrective action taken by the device.

In other embodiments, an external programmer controls the reprogramming under clinician supervision. Upon detection of a decrease in the signal amplitude below a nominal value or below a programmable value selected by a clinician on a primary sensing channel, the device notifies the patient and/or the clinician using suitable warning signals and records suitable diagnostics identifying the drop in the signal amplitude. In one example, the goal is to identify a decrease in the signal amplitude that is not yet below the programmed sensitivity of the implantable device. As such, sensing continues to be normal but if steps are not taken, further deterioration of the sensing signal amplitude could result in a failure to properly sense signals. During a subsequent consultation, the clinician reviews the diagnostic data and then uses the external programmer to run a series of “semi-automatic” tests wherein the implanted device is controlled to sense signals using other sensing configurations available to the device. The clinician examines resulting cardiac signal data sensed using the various configuration and selects a preferred alternate configuration, which is then programmed into the device. Depending upon the capabilities of the implanted device and any external systems used therewith, the clinician may be able to reprogram the device remotely, thereby avoiding the need for the patient to return to the clinician for a reprogramming session.

In still other examples, the entire procedure occurs in-clinic. That is, when the patient is in clinic (or at least under the control of the programmer), the clinician triggers the device to run through its optimization algorithm/procedure and make recommendations for changing the vector as needed.

Still further, various embodiments of the invention can exploit one of more of the following features. The aforementioned capabilities can be performed periodically and automatically to alter the sensing configuration and/or can be performed periodically and automatically but in a “monitor” mode (where the programmed vector is not altered but a report is generated.) Further, selected vectors can be disabled or excluded (such as with ICDs where it is not advisable to enable a unipolar system with one electrode totally outside the heart). In some examples, the “disabled” vectors are analyzed as part of the amplitude assessment but the system cannot reprogram to the vectors. In other examples, the vectors are excluded even from any assessment. Moreover, a nominal sensing amplitude can be specified, which if detected, triggers an assessment automatically or triggers notification to the patient/physician. The nominal sensing amplitude can also be a programmable value (for example, if the initial R wave amplitude is 20 mV, the clinician might want an assessment if the R wave amplitude falls below 10 mV or 5 mV at the time of the periodic evaluation.) Still further, even in circumstances where the signal is large, the overall system can be equipped to allow the clinician to command an evaluation at the time of an office-based evaluation and then select a different sensing vector. Moreover, the overall system can provide the capability for the clinician to electively disable certain configurations from consideration or testing.

As can be appreciated, a wide variety of techniques may be implemented in accordance with the principles of the invention and the foregoing embodiments are merely illustrative.

### BRIEF DESCRIPTION OF THE DRAWINGS

Further features and advantages of the present invention may be more readily understood by reference to the following description taken in conjunction with the accompanying drawings, in which:

FIG. 1 illustrates pertinent components of an implantable medical system having a pacer/ICD equipped to assess sensing issues and then selectively reprogram sensing configurations to address the issues or notify an external system of the sensing problems to allow clinician intervention;

FIG. 2 is a flow chart summarizing exemplary assessment and reprogramming techniques for use with the pacer/ICD of FIG. 1;

FIG. 3 illustrates a device-based embodiment of the technique of FIG. 2 wherein reprogramming is performed by the device itself;

FIG. 4 provides further details regarding the device-based example of FIG. 3 pertaining to sensitivity monitoring and remote notification;

FIG. 5 provides further details of the device-based example of FIG. 4 pertaining to sensitivity monitoring and remote programming;

FIG. 6 illustrates an external programmer-based example of the technique of FIG. 2 wherein reprogramming is performed by an external programmer under clinician supervision;

FIG. 7 illustrates an exemplary diagnostics screen that may be displayed by the external programmer of the technique of FIG. 6;

FIG. 8 illustrates another exemplary diagnostics screen that may be displayed by the external programmer of the technique of FIG. 6;

FIG. 9 provides further details regarding the programmer-based example of FIG. 6 pertaining to an office-based evaluation of signal amplitudes between multiple electrode pairs;

FIG. 10 is a flow chart summarizing the use of an alternative sensing channel to assess sensing issues during “background” processing that may be performed by the pacer/ICD of FIG. 1;

FIG. 11 illustrates an example of the use of primary and alternate sensing channels for use with the technique of FIG. 10;

FIG. 12 is a simplified diagram illustrating the pacer/ICD of FIG. 1 in electrical communication with three leads implanted into the heart of a patient for delivering multi-chamber stimulation and shock therapy;

FIG. 13 is a functional block diagram of the pacer/ICD of FIG. 12, illustrating basic circuit elements that provide cardioversion, defibrillation and/or pacing stimulation in four chambers of the heart, and particularly illustrating components for performing or controlling the various device-based techniques described herein; and

FIG. 14 is a functional block diagram illustrating components of a device programmer for use in programming the pacer/ICD of FIG. 12, and in particular illustrating components for performing the various external system-based techniques described herein.

### DESCRIPTION OF THE PREFERRED EMBODIMENTS

The following description includes the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

### Overview of Implantable System

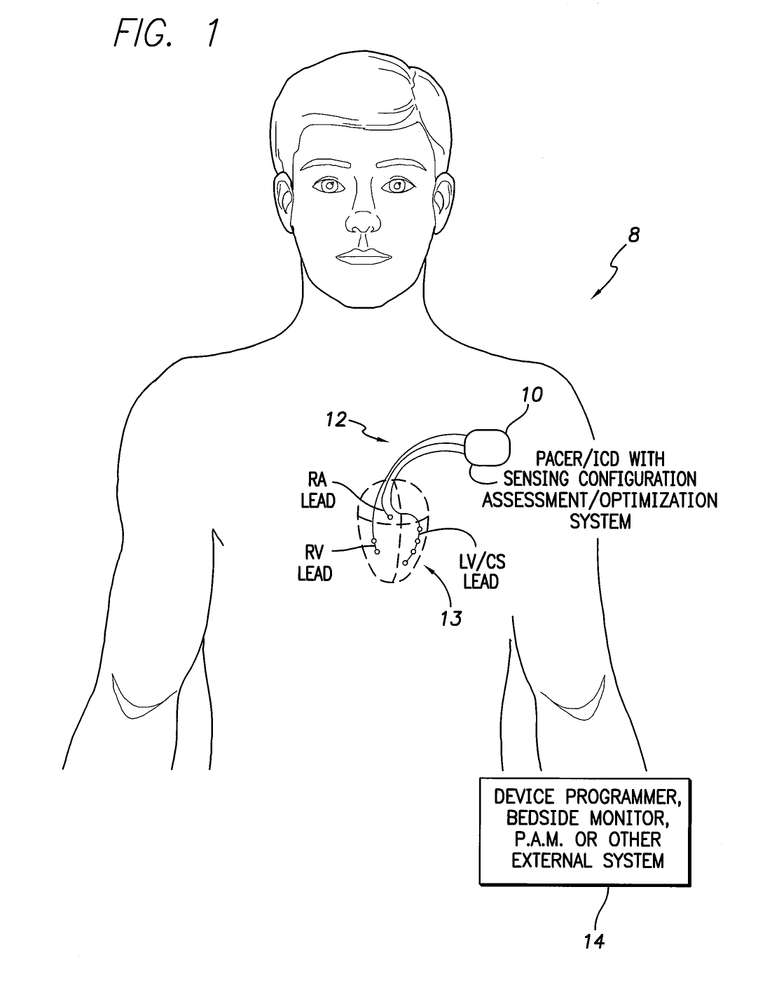

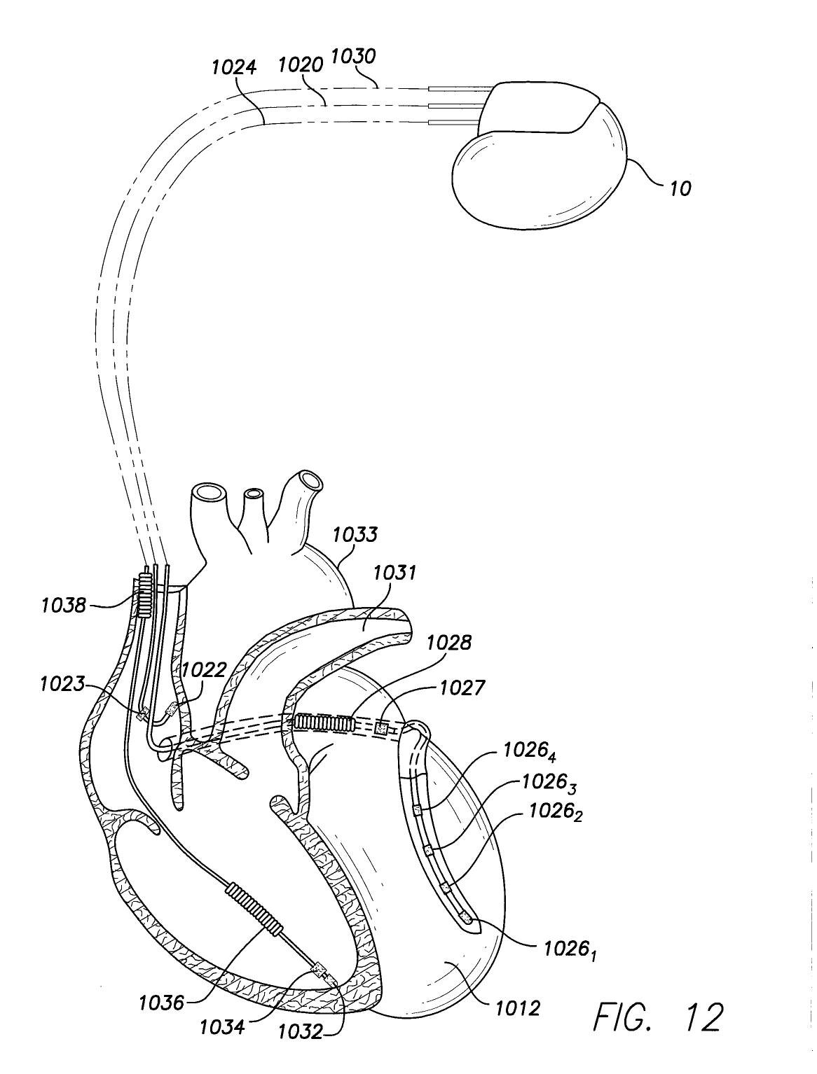

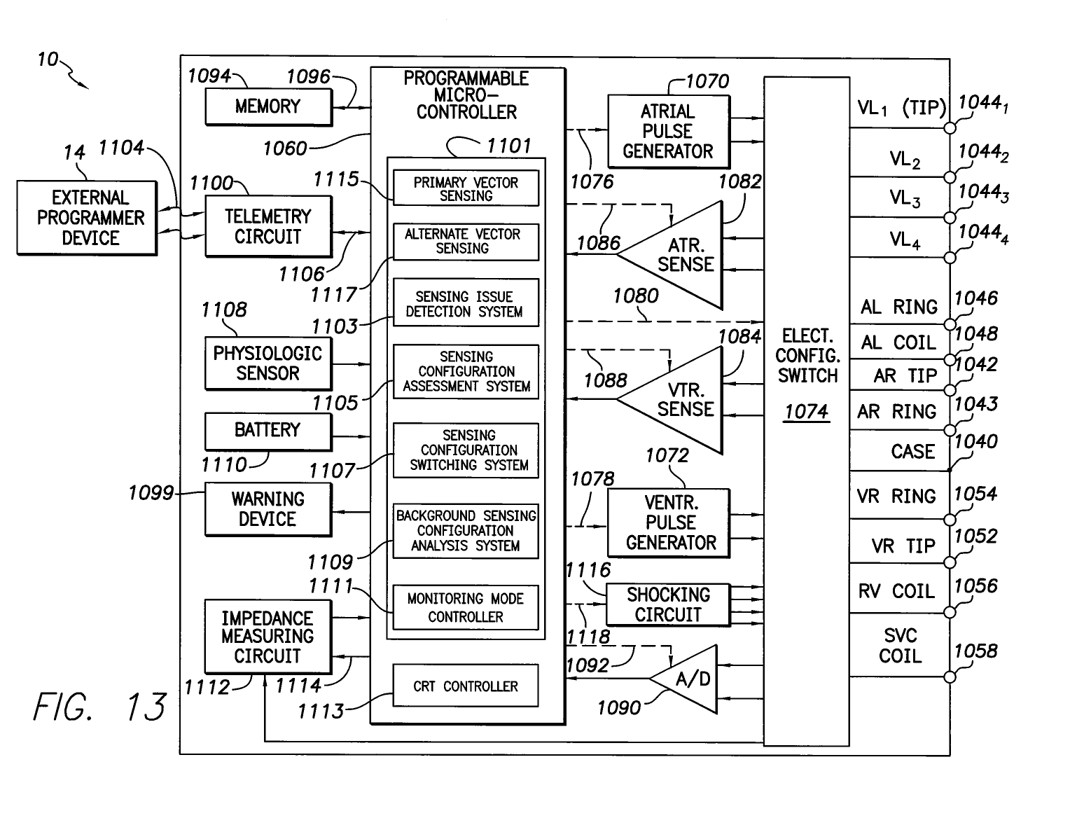

FIG. 1 illustrates an implantable medical system 8 capable of assessing potential sensing issues (particularly a significant drop in signal amplitude) and selectively reprogramming sensing configurations to address the issue. The medical system 8 includes a pacer/ICD 10 or other cardiac rhythm management device (such as a cardiac resynchronization therapy (CRT) device) equipped with one or more cardiac sensing/pacing leads 12 implanted on or within the heart of the patient, including in this example an RA lead, an RV lead, and multi-pole LV lead implanted via the coronary sinus (CS). To illustrate the multi-pole configuration of the LV lead, a set of electrodes 13 is shown distributed along the LV lead. The RV and RA leads are each shown with a bipolar tip/ring electrode pair, though each of those leads may include additional electrodes as well. Still further, the LV lead can also include one or more left atrial (LA) electrodes mounted on the LA via the CS. The various electrodes provide numerous sensing vectors that can be used for sensing cardiac signals within the pacer/ICD on one or more sensing channels. (See FIG. 12 for a more complete and accurate illustration of various exemplary leads and see FIG. 13 for a discussion of the various sense amplifiers and sensing channels.)

In some implementations, the implanted system operates to detect a drop in peak signal amplitudes on a currently-programmed sensing vector over a period of days or weeks and then assesses the sensing vectors to identify an alternative sensing vector that provides acceptable sensing and reprograms the device to automatically remedy the problem (assuming this automatic reprogramming feature has been enabled by the clinician). In other implementations, the device detects a significant drop in the signal amplitude and then transmits appropriate diagnostics information to an external device, such as a programmer 14, which then evaluates or assesses the signal amplitude and either reprograms the pacer/ICD or provides the results of the evaluation to clinician review for subsequent programming. Note that other external devices might instead be used to perform the reprogramming techniques, such as bedside diagnostic monitors, personal advisory modules (PAM) or the like. In some embodiments, the external device is directly networked with a centralized computing system, such as the HouseCall™ system or the Merlin@home/Merlin.Net systems of St. Jude Medical for remote analysis and potential for clinician-authorized programming (remote programming) in systems so equipped.

Warnings to the patient can be generated, when needed, using an internal warning device within the pacer/ICD (such as a vibrating device or a voltage “tickle” device or automatic periodic sounds or beeps generated by the implanted device) or via the beside monitor or PAM. The patient then notifies the clinician or, in some cases, the clinician is automatically notified via networked systems.

In the following illustrative examples, depending upon the particular features being described, some steps/functions are performed by the pacer/ICD, others by the external programmer (or other external device.) Collectively, the pacer/ICD and external programmer device are generally referred to as the system.

### Overview of Reprogramming Techniques

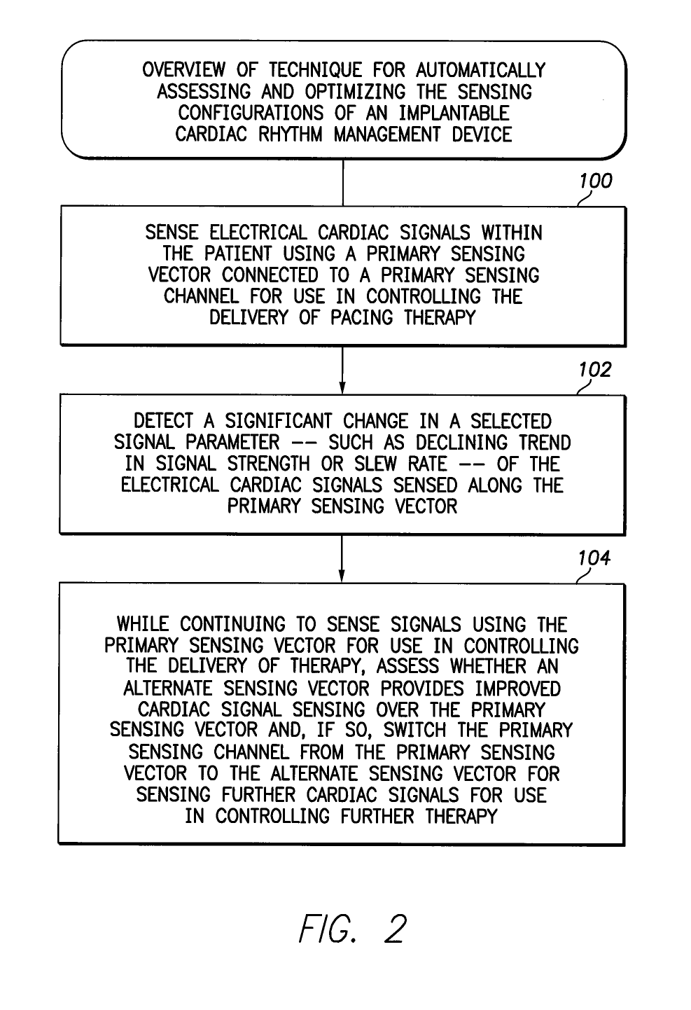

FIG. 2 broadly summarizes a general technique that can be exploited by the system of FIG. 1 (or other suitably-equipped medical systems) for reprogramming sensing vectors. Beginning at step 100, the system senses electrical cardiac signals within the patient using a primary sensing vector connected to a primary sensing channel for use in controlling the delivery of pacing therapy. That is, one or more sensing vectors are exploited between or among the various electrodes of the lead system for use in sensing electrical cardiac signals, such as IEGM signals. Separate primary sensing vectors may be defined for the atria and the ventricles. Still further, separate primary sensing vectors may be defined in the LV and RV. In one example, a primary LV sensing vector might be a bipolar vector defined between an LVtip electrode and a first LVring electrode. For clarity in describing the invention, unless otherwise noted, only a single primary sensing vector will be described.

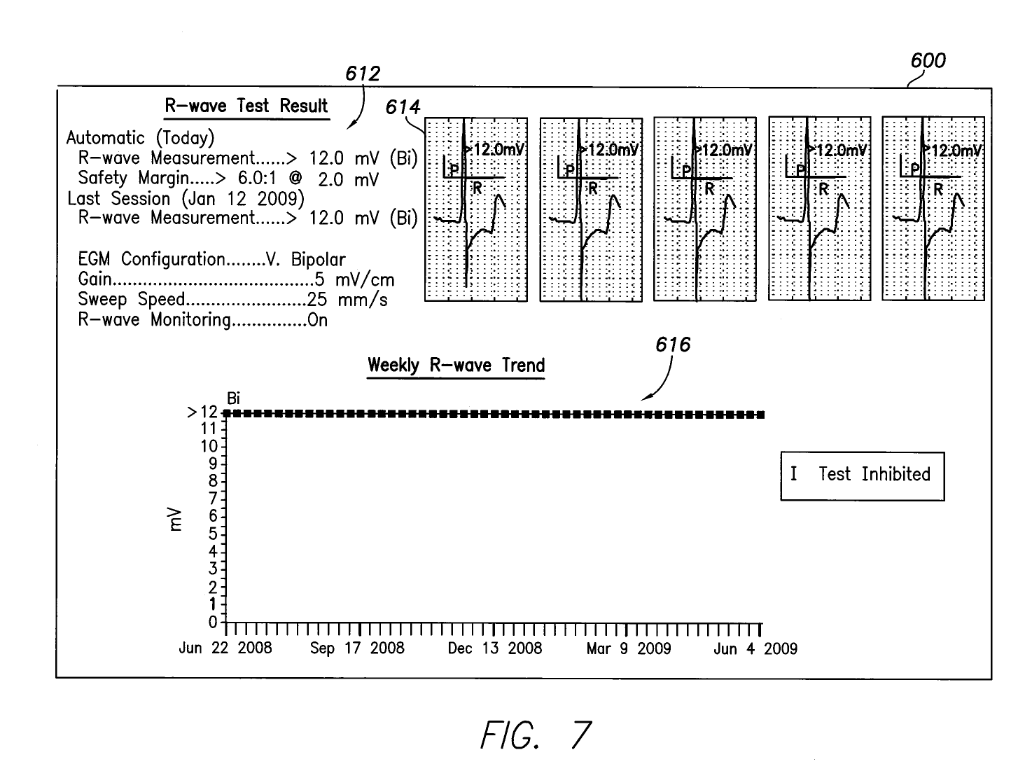

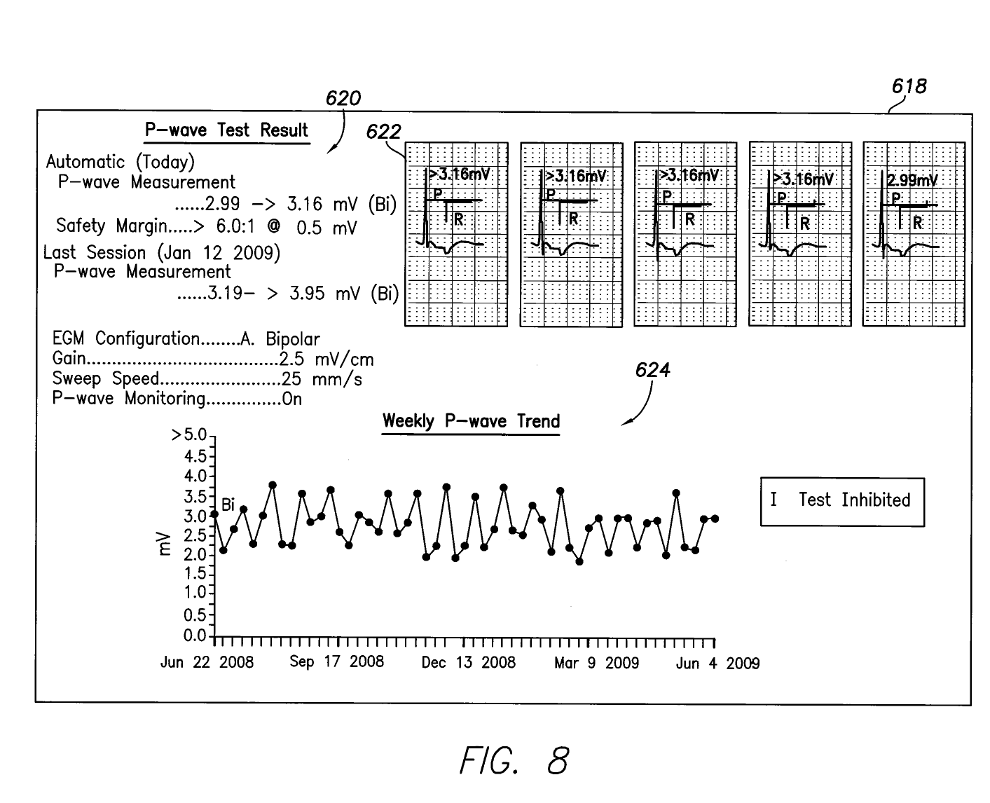

At step 102, the system detects a significant change in a selected signal parameter—such as declining trend in peak signal strength or peak slew rate—of the electrical cardiac signals sensed along the primary sensing vector. As noted above, the term trend refers to generally long-term changes in signal parameters, i.e. changes occurring over at least several cardiac cycles rather than during a single cardiac cycle. As can be appreciated, during individual cardiac cycles, there can be significant changes in amplitude, slew rate or other signal parameters. These are not the type of changes detected at step 102. Rather, at step 102, changes occurring over a longer period of time—minutes, hours, days, weeks or even longer intervals—are typically detected, such as changes in average signal peak amplitude over a period of months. This is discussed in greater detail below, particular with reference to FIGS. 7 and 8.

As one example of the operation of step 102, the system can be programmed to detect a drop in the peak amplitude (or magnitude) of the cardiac signals (such as by comparing against a predefined threshold value or a physician-programmed value) over time, of the type potentially leading to the undersensing of QRS complexes or other cardiac events if the drop in the signal amplitude continues to progress. That is, a sensing issue is detected that is indicative of a potential (but not yet manifest) sensing problem. (Note that the term amplitude, as used herein, is generally synonymous with magnitude, i.e. it refers to the size of the signal, which might be positive or negative, depending upon the polarity of the signal.) In other examples, rather than assessing trends in signal amplitude, other trend parameters are additionally or alternatively assessed, such as trends in slew rate, which can be defined as voltage change per time (dV/dt).

At step 104, while the device continues to sense signals using the primary sensing vector for use in controlling the delivery of therapy, the system assesses whether an alternate sensing vector provides improved cardiac signal sensing over the primary sensing vector and, if so, switches the primary sensing channel from the primary sensing vector to the alternate sensing vector for sensing further cardiac signals for use in controlling further therapy (assuming this automatic reprogramming feature has been enabled by the clinician.) For example, the device can systematically assess the signal amplitudes for all other candidate sensing vectors using the available electrodes of the lead system (e.g., LVtip—LVring1, LVtip—LVring2, LVtip LVring3, LVring2—LVring3, LVtip—RVtip, etc.) The system identifies the vector that provides the best sensing amplitude (or which satisfies other criteria) and the reprograms the implantable device accordingly. That is, in one example, the “best” sensing vector is the vector providing the largest signal amplitude. In other examples, other criteria can be used to choose among alternate vectors. For instance, if multiple vectors are deemed better than the primary vector or perhaps not as large as the primary vector but still adequate with other beneficial features such as a narrower bipole, the system may be programmed to choose the vector with the closest bipole among various candidate vectors, even if that vector does not have the largest signal or even a signal as large as the originally programmed primary vector.

As noted, the system continues to sense using the primary sensing vector connected to the primary sensing channel while alternative sensing vectors are examined in the background using an alternative sensing channel, so that therapy is not interrupted. Any such background processing of cardiac signals on alternative channels can be triggered upon detection of a sensing issue on the primary sensing channel or, in some embodiments, can be performed more or less continuously to monitor for an alternative sensing vector that might be better than the current sensing vector.

In enabling this capability, the physician or clinician can selectively exclude specific electrode-pair combinations that would be inappropriate for a given patient/device even if that signal amplitude might provide the largest signal. For example, this might entail exclusion of all unipolar configurations involving one electrode in the heart and the other electrode being the housing of the pulse generator and/or any other subcutaneous electrode in ICD systems.

Thus, these techniques allow the sensing circuit of the implanted device—either under the control of the clinician caring for the patient or under the control of the device itself—to automatically screen all (or some of) the potential combinations of electrode pairs to identify the best signal vector.

The general technique of FIG. 2 broadly covers the following exemplary scenarios. In one scenario, the signal amplitude decreases to unacceptable levels and this is discovered at the time of a routine follow-up evaluation or because it has triggered inappropriate therapy that brought the patient back to the clinician's office or clinic:
Inappropriate therapy could be T-wave oversensing triggering inappropriate shocks because the system labels a normal rhythm as VT or VF.
Failure to recognize a pathologic arrhythmia such as AF and thus failure to mode switch; also failure to engage the diagnostics such that the diagnostics then provide inaccurate information.
Failure to recognize a pathologic arrhythmia such as AF with the intermittent sensed complexes being labeled atrial premature beats triggering a progressive increase in the atrial paced rate (even though atrial pacing is ineffective because of the underlying AF) and ventricular paced rate to the maximum AF Suppression rate. Sustained high rates, be it from pacing or the intrinsic rhythm if it is fast, can induce heart failure.
Physician initiates the sensing configuration procedure/algorithm where the pulse generator systematically assesses various combinations of electrodes and reports these results to the clinician with a recommendation as to which pair gives the best signal.
The physician can then elect to program this configuration or choose a different configuration. This is totally under the control of the clinician.

In another exemplary scenario, the device itself periodically assesses the signal amplitude. If the device recognizes a signal amplitude below a physician-selected or device-programmed nominal value, the device itself automatically searches for the electrode pair that provides the largest signal amplitude. In a preferred implementation, this evaluation is conducted using an alternate sensing channel, such that, during the testing period, the actual sensing behavior of the device is not affected; this avoid potentially inappropriate behavior associated with temporarily sensing from a vector that is suboptimal. In this scenario, the device:
Triggers an alert to notify the patient and/or triggers an alert that is “silent” so the patient is not aware of it but the clinician is notified.
Initiates a procedure/algorithm that sequentially scans through the available combination of electrode pairs with respect to signal amplitude.
Generates a summary of the evaluation results available the next time that the implantable device is evaluated in clinic, allowing the clinician to make appropriate adjustments using the programmer. These results can also be downloaded to the clinician via Merlin.net or a similar remote monitoring program; when remote programming is available, the clinician may make the appropriate adjustments without the need to bring the patient to the clinic.
If the signal amplitude is very low, a sequential search of different electrode combinations may identify a better signal amplitude. The system then switches to that sensing configuration. At the time of the switch, it also generates an alert either so that the patient is aware of it or simply to bring this to the attention of the clinician.
When the automatic algorithm/procedure scans the different electrode combinations, it stores resulting information so that the device can report the information (a) at the time of a follow-up evaluation, (b) via the Internet [Merlin.net]. Preferably, the device reports all the results of the evaluation even if it was set to automatic and selected the largest signal between two pairs of electrodes.

In yet another exemplary scenario, a full-manual assessment/reprogramming procedure is provided. That is, the sensing vector analysis is initiated by the clinician with the programmer (or remotely), and then reprogramming is controlled via the external programming system. More specifically, the clinician initiates the overall assessment by entering an appropriate command into an external device, which then generates and sends various specific commands to the implanted device for controlling the device to perform a series of tests. In preferred examples, the assessment algorithm/procedure/system is contained in the implantable device and the programmer simply requests that the algorithm be run by the device. In other examples, the algorithm/procedure/system for selecting and sequencing vectors is built into the programmer, and the whole process is under the control of the programmer, but the algorithm is directed automatically by the programmer, rather than by individual explicit steps by the clinician.

These and other exemplary scenarios will be described in greater detail with reference to the remaining figures.

### Pacer/ICD-based Automatic Sensing Vector Reprogramming

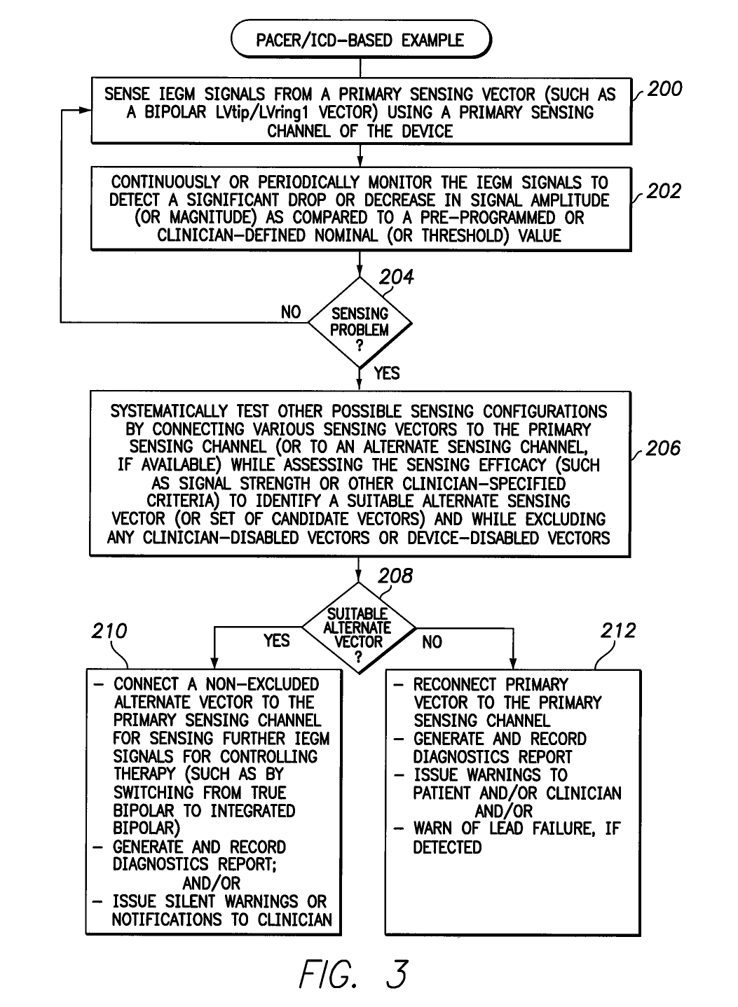

FIG. 3 provides further details of an example wherein the functions are performed by the pacer/ICD (which has been previously enabled by a clinician to perform these functions.) Beginning at step 200, the pacer/ICD senses IEGM signals from a primary sensing vector (such as a bipolar LVtip—LVring1 vector) using a primary sensing channel of the device. At step 202, the pacer/ICD continuously or periodically (or subject to clinician command) monitors the sensed IEGM signals to detect a significant drop in signal amplitude (or magnitude) as compared to a pre-programmed or clinician-defined threshold value. Note that a drop in signal amplitude can ultimately result in such problems as oversensing of cardiac signals; undersensing of cardiac signals; delivery of inappropriate shocks; failure to deliver appropriate shocks; failure to detect a pathological arrhythmia such as AF, VF or VT; failure to mode switch when appropriate; failure activate diagnostics when appropriate; failure to detect AF with the intermittent sensed complexes being labeled atrial premature beats triggering a progressive increase in atrial paced rate. However, it should be understood that these problems need not be specifically detected by the device. That is, the drop is sensing amplitude detected at step 202 need not be so great as to have already triggered these problems. Indeed, the goal is to remedy the drop in signal amplitude before such problems arise.)

More specifically, low signal amplitudes may be detected by the pacer/ICD by using preprogrammed threshold values indicative of minimum acceptable signal amplitudes. If the electrical cardiac events to be detected (such as QRS-complexes on a ventricular channel) have peak amplitudes that fall below the minimum threshold, then a “significant drop” in the signal is thereby detected. Typically, different thresholds are provided for ventricular channels as opposed to atrial channels.

Assuming a decrease in signal amplitude below a preset or nominal value has been detected (at step 204), then the pacer/ICD, at step 206, systematically tests other possible sensing configurations by connecting various sensing vectors to the primary sensing channel (or preferably to an alternate sensing channel) while assessing the sensing efficacy (such as signal strength, amplitude or magnitude, slew rate, bipolar spacing, interelectrode distance or other clinician-specified criteria) to identify a suitable alternate sensing vector or set of candidate vectors (and while excluding any clinician-disabled vectors or device-disabled vectors.) For example, if the LVtip—LVring1 vector is currently the primary sensing vector and a drop in signal amplitude below a preset or nominal value is detected, the pacer/ICD then sequentially and systematically connects all other possible ventricular sensing vectors (such as LVtip—LVring2, LVtip—LVring3, LVring2—LVring3, etc.) to an alternate ventricular sensing channel to identify any sensing vectors that might provide a sufficiently large signal amplitude allowing maintenance of a sufficient sensing safety margin thus minimizing the chance of a potential sensing problem. In a particular example where the LV lead is a quadrapole (or “quadpole”) lead similar to SJM's Quartet® lead, the various programmable sensing vectors are as shown in Table I.

TABLE I

| Unipolar | Bipolar |
|---|---|
| LVtip-Can | LVtip-LVring1 |
| LVring1-Can | LVtip-LVring3 |
| LVring2-Can | LVring1-LVring3 |
| LVring3-Can | LVring2-LVring1 |
| | LVring2-LVring3 |
| | LVring3-LVring1 |

As noted above, in some examples, some of the vectors are disabled or excluded by the clinician (or simply based on device type.) In this regard, there may be situations where the unipolar configuration options (when involving one electrode physically outside the heart) can be precluded from being either tested and/or utilized. An example of a device-type exclusion is to exclude certain vectors for all models of a type of device, such as for all models of ICDs. Also, insofar as clinician-specified criteria, the system can employ an algorithm/procedure that uses criteria other than purely signal quality/amplitude to choose among alternate vectors. For example, if multiple vectors are deemed better than the primary vector, the algorithm/procedure may choose the acceptable vector with the closest bipole among the acceptable vectors, even if that vector were not the one with the absolute largest signal. In still other examples, the system chooses the electrode pair having the shortest interelectrode distance as long as the amplitude and slew rate are adequate for sensing.

Assuming that at least one acceptable alternate sensing vector is detected (at step 208), then the pacer/ICD, at step 210, automatically connects the best of the (non-excluded) alternate vectors to the primary sensing channel for sensing further IEGM signals for use in controlling therapy. That is, in this illustrative embodiment, the pacer/ICD automatically reprograms its sensing configuration to use the alternate vector. The pacer/ICD can also generate and record a diagnostics report and issue silent warnings or notifications to clinician, if so programmed. The diagnostic report preferably identifies and documents the original signal amplitude value that triggered the search for an alternate sensing vector and also provides diagnostic information indicative of the efficacy of the selected alternate channel. In some cases, two or more alternate sensing vectors might be detected that are preferable to the current primary sensing vector. If so, the pacer/ICD determines which of the alternate sensing vectors is optimal by, e.g., assessing the amplitudes of signals on the channel, the amount of noise, etc. The device then reprograms its sensing configuration using the sensing vector deemed to be optimal. This algorithm/procedure can be enabled in either a monitor mode (where it reports the potential changes in sensing configuration but does not program any changes) or an active mode (where it automatically makes the change to an electrode configuration that results in a larger signal.)

If no suitable alternate vector is found, then at step 212, the pacer/ICD reconnects the primary vector to the primary sensing channel (if needed) and then generates and records a diagnostics report for clinician review. The device also issues warnings to the patient and/or clinician indicating that there is an on-going sensing issue that the device itself was unable to remedy. Preferably, the clinician then promptly consults with the patient and then reviews and corrects the problem, which might require repositioning or replacing one or more leads.

As already noted, automatic reprogramming of sensing vectors by the device is preferably performed only if the clinician has enabled that feature. In this regard, automatic switching from a bipolar to a unipolar vector (in pacemakers and potentially on the atrial lead of an ICD) without the clinician being able to assess issues such as myopotential susceptibility (especially with autosensing) might be regarded by at least some clinicians as risky (in the absence of a clear failure of the originally programmed vector) and hence not enabled. Hence, as already discussed, it may be appropriate to disable or exclude certain sensing vectors from assessment or reprogramming,

Automatic reprogramming might be preferred, however, in systems having multiple electrodes in the same chamber in relatively close proximity to one another. In systems without multiple electrodes in the same chamber (in relatively close proximity to one another), the clinician might prefer to limit the automatic reprogramming to switching between true bipolar and integrated bipolar (especially for RV ICD leads.) (Note that at least some current systems use multiple electrodes but have only a single sensing configuration. Some of the embodiments of the present invention take advantage of multiple electrodes which are each capable of being incorporated into a bipolar pair for sensing.)

Integrated bipolar sensing is discussed in U.S. Pat. No. 6,947,794 to Levine, entitled “System and Method with Improved Automatic Testing Functions for Defining Capture Thresholds”, U.S. Pat. No. 6,766,197 also to Levine, entitled “System and Method with Improved Automatic Testing Functions for Automatic Capture Verification,” and U.S. Pat. No. 7,610,090 to Hofstadter, et al., entitled “Implantable Medical Device with Automatic Sensing Adjustment.” In some examples, automatic reprogramming might be limited to reprogramming among certain specific vectors designated by the clinician or, as already noted, reprogramming might be limited to circumstances where a complete lead failure has been detected.

Still further, note that the likelihood of finding an acceptable vector when using quadpole leads without having to revert to a “unipolar” configuration is very high. This is not necessarily the case with systems that have a single bipolar lead in the ventricle and a single bipolar lead in the atrium. For such systems, it might be appropriate to exploit Combipolar sensing techniques. With Combipolar sensing, the system uses two unipolar leads—one in the atrium and one in the ventricle. Atrial sensing is Atip-Vtip, Vent sensing is Vtip-Case. That is, signals seen on both channels are ventricular signals; signals seen only on the atrial channel are atrial signals. Thus, in at least some embodiments of the invention, automatic programming to the “unipolar configuration” is employed if this also engages the Combipolar algorithm/procedure/system. For a more complete description of Combipolar systems, see U.S. Pat. No. 5,522,855 to Hoegnelid.

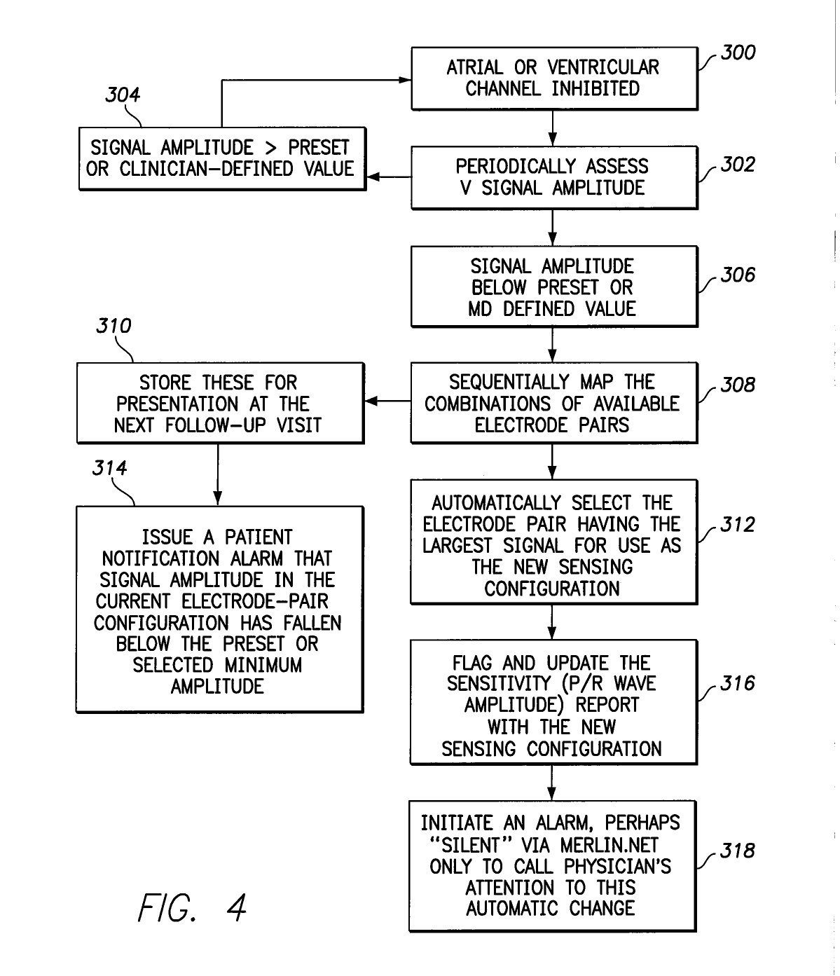

Turning now to FIG. 4, further details are provided pertaining to a pacer/ICD-based implementation, particularly directed to sensitivity monitoring and clinician notification. That is, these are techniques that may be performed by a suitably-equipped pacer/ICD in addition to, or as an alternative to, the techniques of FIG. 3. Beginning at step 300, the implanted device operates in a mode (or switches to a mode) wherein the atrial or ventricular channel is inhibited. Typically, for a ventricular channel sensing test, the ventricular channel should be inhibited. For an atrial channel sensing test, the atrial channel should be inhibited. While operating in the inhibited mode, at step 302, the device periodically assess the ventricular signal amplitude (i.e. the amplitude of R-waves.) Assuming the amplitude exceeds a preset or clinician (i.e. MD) defined value representative of an acceptable amplitude, as determined at step 304, then processing returns to step 300. If the signal amplitude falls below the threshold value, at step 306, then processing proceeds to step 308 wherein the device sequentially maps the various combinations of available electrode values. By mapping, it is meant that the device senses and stores sample signals using a set of available electrode pairs or vectors in some systematic fashion.

Then, depending on its programming, the device stores, at step 310, the accumulated data for presentation to the MD at a next in-office follow-up session and/or the device, at step 312, automatically selects the electrode pair having the largest signal for use as the new sensing configuration. This may be performed by resetting the appropriate programmable parameter within the device that defines the current sensing configuration. If step 310 is performed then, at step 314, a notification alarm signal is issued to the patient indicating that the signal amplitude in the current electrode pair configuration has fallen too low. If step 312 is performed then, at step 316, the device flags and updates a sensitivity (P/R wave amplitude) report maintained by the device to note the change in sensing configuration (made at step 312.) Then, at step 318, the device issues an alarm, perhaps silent, via Merlin.net to notify the MD of the automatic change in the sensing value made at step 312.

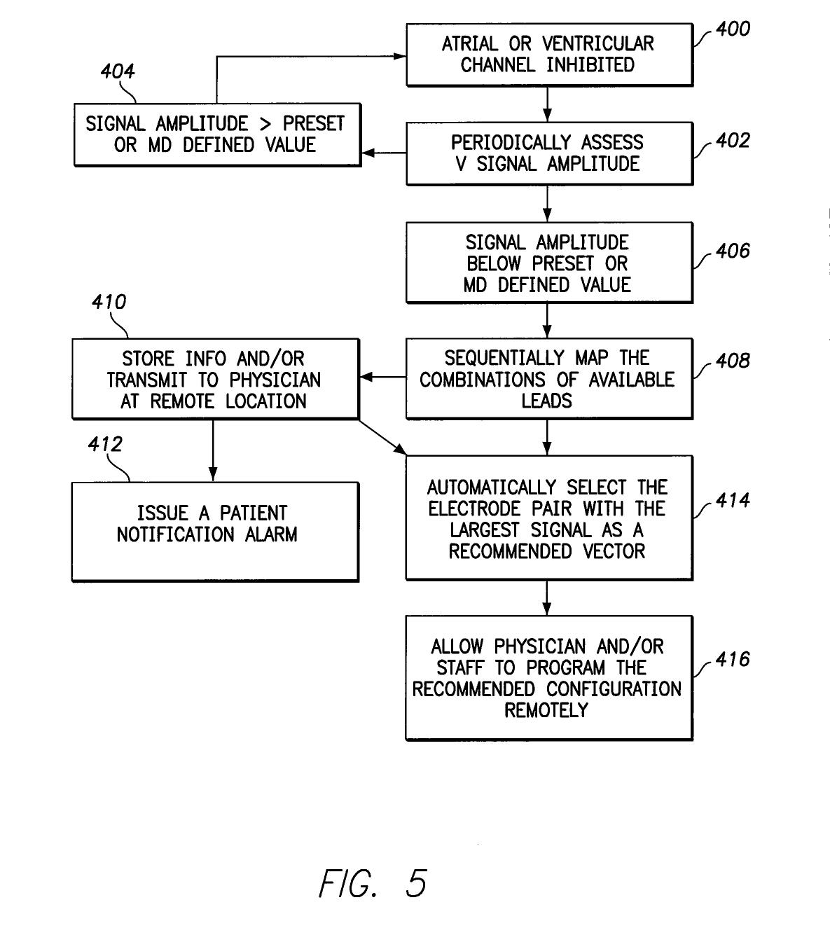

Turning now to FIG. 5, still further details are provided pertaining to an exemplary pacer/ICD-based implementation, particularly directed to sensitivity monitoring and remote programming. These again are techniques that may be performed by a suitably-equipped pacer/ICD in addition to, or as an alternative to, the techniques of FIGS. 3 and 4. (Some of these steps are the same as in FIG. 4 and hence will be only briefly described.) Beginning at step 400, the device again operates in a mode wherein the atrial or ventricular channel is inhibited. While operating in an inhibited mode, at step 402, the device periodically assesses signal amplitudes and compares to a preset or physician prescribed value. If the amplitude exceeds the threshold, at step 404, then processing returns to step 400. If the signal amplitude falls below the threshold value, at step 406, then processing continues to step 408 wherein the device sequentially maps the various combinations of available electrode values.

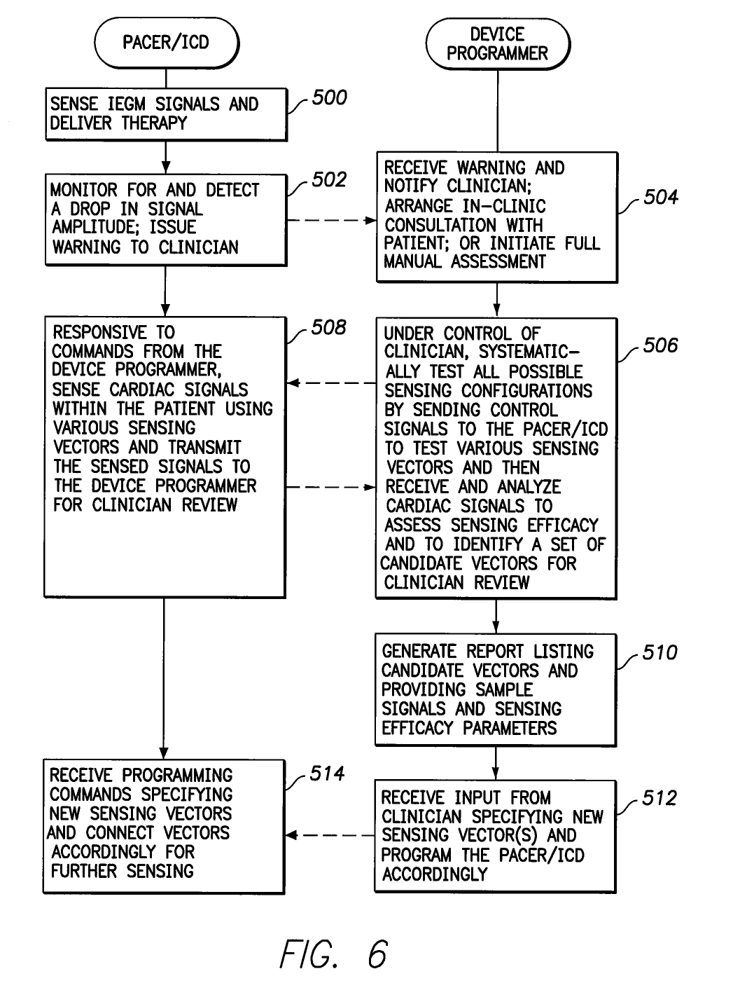

Then, at step 410, the device stores the accumulated data and/or transmits the data to the physician at a remote location (using any suitable communication network such as Merlin.net) and, at step 412, issues a patient notification alarm. Also, at step 414, the device selects the electrode pair with the largest signal as a “recommended” sensing vector. Note that, unlike the embodiment of FIG. 4, the device in this example does not automatically reprogram its operation to use that sensing vector. Rather, at step 414, the clinician (and/or their staff) reviews data provided by the pacemaker and can then reprogram the device based on the recommended configuration (or to use a different configuration.) The review can be performed at a remote location. That is, in this embodiment, the patient need not return to the clinic. The physician (or staff) reviews data provided by the pacemaker and reprograms the device remotely. Alternatively, the physician can instead have the patient return to the clinic for further consultation (see, generally, FIGS. 6-9), particularly if the physician is not entirely satisfied by the device-recommended sensing vector.

### In-Clinic Programmer-Based Sensing Vector Reprogramming

Turning now to FIGS. 6-9, reprogramming techniques will be described with reference to an in-clinic example performed under clinician supervision. This may be regarded as “semi-automatic reprogramming.” At step 500 of FIG. 6, the pacer/ICD senses IEGM signals using a currently-programmed set of sensing vectors and delivers therapy in accordance with otherwise conventional techniques. At step 502, the pacer/ICD monitors for and detects a drop in signal amplitude and issues a warning to the clinician. Warnings may be relayed using any suitable techniques. In one example, an internal warning is generated within the patient using a tickle or vibrational warning device, which prompts the patient to position a PAM near the implanted device, while another warning is a series of audible beeps. Information specifying to the sensing issue is transmitted to the PAM and then relayed to the clinician (or other appropriate personnel) via systems such as the aforementioned Merlin@home/Merlin.Net systems. In any case, the warnings are eventually routed to an external device programmer operated under the control of a clinician, at step 504, for use during an in-clinic consultation with the patient. Alternatively, at step 504, the clinician can simply initiate a “full manual” assessment, even in the absence of any detected drop in signal amplitude.

During the in-clinic consultation, the programmer systematically tests all possible sensing configurations, at step 506, under the control of the clinician by sending control signals to the pacer/ICD to test or map various sensing vectors. At step 508, in response to commands from the programmer, the pacer/ICD senses cardiac signals within the patient using various sensing vectors and then transmits the sensed signals to the device programmer for clinician review. These may be the same tests described above in connection with step 206 of FIG. 3, but performed under the control of the programmer while under clinician supervision. The programmer receives and analyzes the cardiac signals (also at step 506) to assess sensing efficacy and to identify a set of candidate vectors for clinician review.

The procedure of steps 504 and 506 continues until all sensing configurations to be tested have been tested and the results compiled. Depending upon the number of electrodes on the various leads there might be a fairly large number of sensing vectors to be tested. If so, various techniques may be exploited for reducing the total number of sensing tests to be performed. See, e.g., U.S. patent application Ser. No. 12/703,069, filed Feb. 9, 2010, of Rosenberg et al., entitled “Systems and Methods for Optimizing Multi-Site Cardiac Pacing and Sensing Configurations for use with an Implantable Medical Device.”

At step 510, the device programmer generates a report listing candidate sensing vectors and providing sample signals and various sensing efficacy parameters (such as values representative of noise levels, signal amplitudes, etc.) In one example, the following table of results is presented (wherein the signal amplitude values are R-wave amplitudes as measured by the implanted device):

TABLE II

| Sensing Vector | Signal Amplitude | |
|---|---|---|
| RVtip-RVring | 2.1-2.3 | mV |
| RVtip-RVcoil | 4.2-5.8 | mV |
| RVtip-LVtip | 8.9-11.2 | mV |
| RVring-LVring3 | >12.0 | mV |

An exemplary display screen 600 is presented in FIG. 7 for an R-wave amplitude test for a ventricular channel sensing vector. Within the display, the numerical results of the signal amplitude test are shown by way of table 612. In addition, a graphic display 614 of five consecutive QRS complexes (or R-waves) is provided, as shown. In this particular example, the QRS complexes are clearly defined with good peak amplitudes, indicating that this particular sensing vector provides effective ventricular sensing. If the appropriate data is available, a weekly trend 616 of R-wave amplitudes can also be displayed. (Alternatively, more frequent or less frequent measurements can be displayed over shorter or longer periods of time.) Typically, this data is only available if the particular ventricular sensing vector in question has been in use by the device during the preceding weeks such that the trend data can be recorded by the device. For most ventricular sensing vectors being tested, the vector will not have been in use previously and hence no trend data will be available and the weekly trend graph will be omitted. Also note that, in this particular example, the weekly trend in data shows consistently high R-wave amplitudes indicative of a properly functioning ventricular sensing channel. It should be understood that, in cases where there is a ventricular channel low amplitude signal, the amplitude values will likely be trending much lower.

Insofar as regulating the sampling of measured parameters, see U.S. Pat. No. 6,366,812 to Levine et al., entitled “Implantable cardiac Stimulation Device and Method for Self-Regulating Sampling of Measured Parameters” and U.S. Pat. No. 6,129,746 also to Levine et al., entitled “Implantable Cardiac Stimulation Device with Dynamic Self-Regulation of the Frequency of Automatic Test Functions and the Recordation Thereof.”

In yet another implementation, each vector is given its own graphic symbol and the graphic display reports the stability and/or fluctuations of different electrode pairs, providing a relatively long term trend for signal amplitude using different symbols for each electrode pair.

Another exemplary display screen 618 is presented in FIG. 8. This display presents results of a P-wave amplitude test for an atrial channel sensing vector. Within the display, the numerical results of the signal amplitude test are shown by way of table 620. In addition, a graphic display 624 of the most recent set of five consecutive P-waves is provided, as shown. In this particular example, the P-waves are clearly defined with good peak amplitudes, indicating that this particular sensing vector provides effective atrial sensing. As with the R-wave display discussed above, if the appropriate data is available, a trend 624 of P-wave amplitudes can be displayed. This can be hourly, daily, weekly or some other unit of time. Again, this data is typically only available if the particular atrial sensing vector in question has been in use by the device during the preceding weeks such that the trend data can be recorded by the device. For most atrial sensing vectors being tested, the vector will not have been in use previously and hence no trend data will be available. Also note that, in this particular example, the weekly trend in data shows that the P-wave amplitudes have been relatively stable and large which in conjunction with the known programmed sensitivity value is indicative of a properly functioning atrial sensing channel. The implication is that this represents normal function. As with the ventricular example discussed above, it should be understood that, in cases where there is a drop in the atrial channel sensing signal, the observed amplitude values would likely be trending much lower. (Note that, if there were signals that were smaller than the programmed sensitivity setting, these would not have been detected and would not be displayed.)

Returning to FIG. 6, at step 510, the programmer receives input from the clinician specifying new sensing vector(s) and programs the pacer/ICD accordingly. At step 512, the pacer/ICD receives the programming commands specifying the new sensing vectors, reprograms its components accordingly and then begins sensing cardiac signals using the new vectors and delivering pacing or other forms of therapy accordingly.

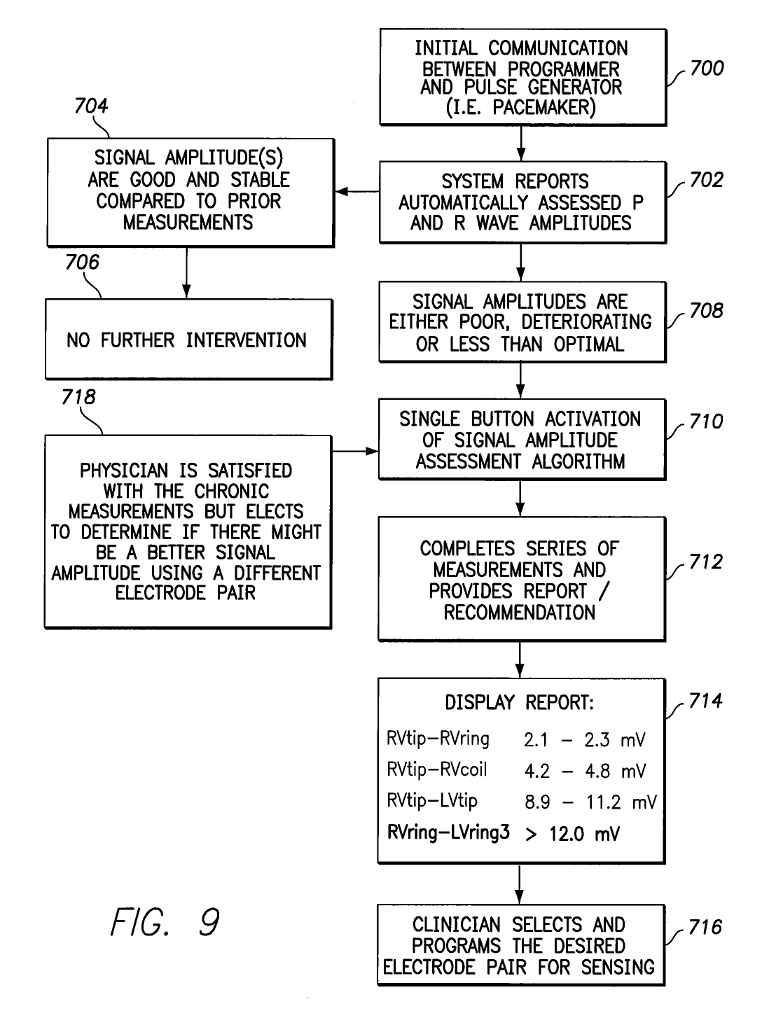

Turning now to FIG. 9, still further details are provided pertaining to an exemplary programmer-based implementation, particularly directed to evaluating signal amplitudes between multiple electrode pairs. That is, these are techniques that may be performed by a suitably-equipped programmer in addition to, or as an alternative to, the techniques of FIG. 6. Beginning at step 700, the programmer initiates communication with the pacer/ICD (i.e. an implanted “pulse generator” device) and retrieves stored data such as P-wave and R-wave amplitudes that were automatically assessed by the pacer/ICD. At step 702, the programmer generates a report listing P-wave and R-wave amplitudes. If at step 704 the amplitudes are found by the clinician to be “good” and “stable” as compared to prior measurements, then no further intervention is required (step 706.) However, if at step 708, the signal amplitudes are found to be poor, deteriorating and/or less than optimal, then step 710 is performed wherein a signal amplitude assessment procedure is initiated (such as the one described in step 506 of FIG. 6, discussed above). Preferably, this test is configured as a “single button” assessment that requires relatively little clinician input or supervision. See, again, the patent application of Rosenberg et al., which describes other “one-step” or “single button” tests. These single button tests are (at least somewhat) analogous in overall concept to the QuickOpt™ procedures described in the above-sited patent applications, where an optimization procedure is initiated by a command on the screen and then the programmer automatically runs through a variety of tests associated with various temporary programming commands, takes the appropriate measurements and then reports the results to the clinician by a display on the programmer screen. The clinician can then accept and program the recommended settings or choose yet a different set of parameters.

At step 712, the programmer completes its series of measurements and provides reports (such as the reports described above) and recommendations as to preferred or optimal sensing vectors. The reports are displayed for clinician review at step 714. Some exemplary data is provided in step 714. Note that the first value represents the measured amplitude on a currently programmed electrode pair. The last value (displayed in bold) represents the recommendation of the programmer for the preferred or optimal sensing configuration. At step 716, the programmer inputs clinician selections of new sensing vectors and reprograms the pacer/ICD to use the selected configurations.

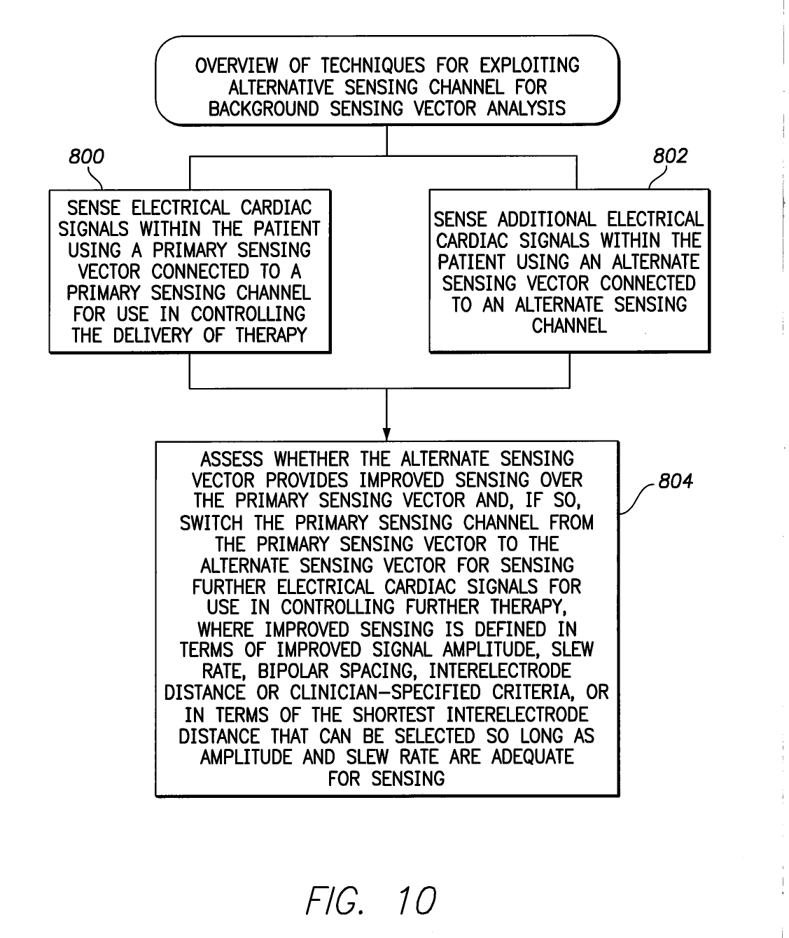

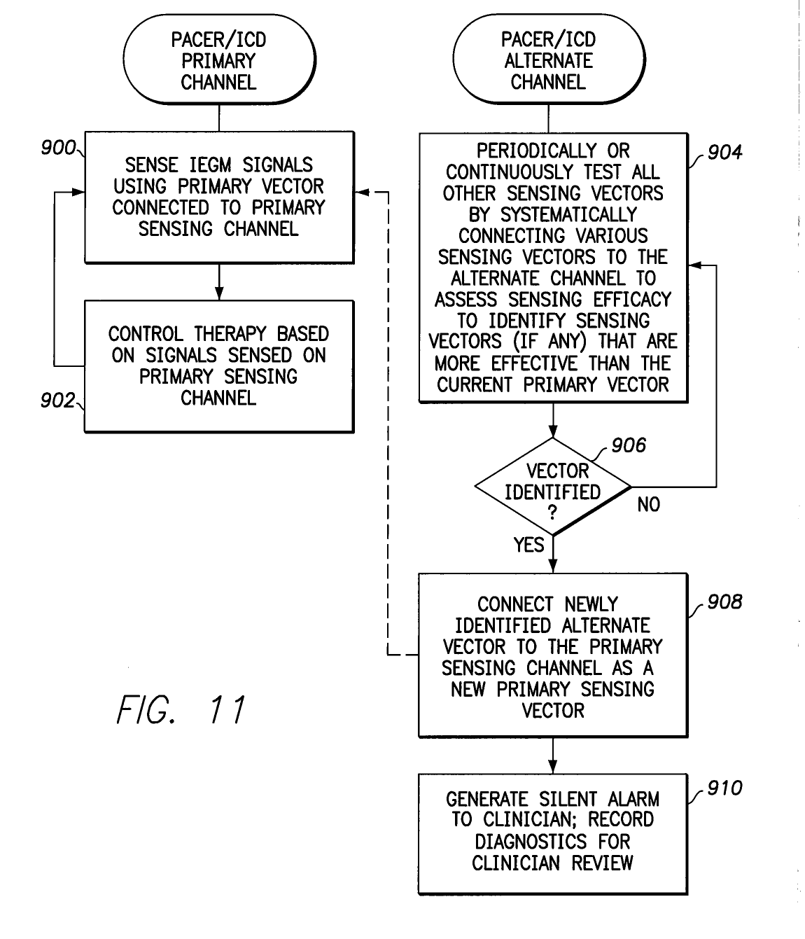

The various techniques described thus far can exploit a form of “background” processing whereby the pacer/ICD continues to sense signals on one or more primary sensing channels, while the device uses an alternative sensing channels to periodically or continuously monitor for a different sensing vector that might provide a larger signal amplitude providing a greater sensing safety margin at the current programmed sensitivity setting. This is described summarized independently with reference to FIGS. 10 and 11.

### Background Analysis Using Alternate Sensing Channel

FIG. 10 broadly summarizes the techniques that exploit an alternative sensing channel for performing a background analysis of sensing vectors, discussed above. Beginning at step 800, the pacer/ICD senses electrical cardiac signals within the patient using a primary sensing vector connected to a primary sensing channel for use in controlling the delivery of therapy. Substantially concurrently or simultaneously, at step 802, the pacer/ICD senses additional electrical cardiac signals within the patient using an alternate sensing vector connected to an alternate sensing channel. At step 804, the pacer/ICD then assesses whether the alternate sensing vector provides improved sensing over the primary sensing vector and, if so, switches the primary sensing channel from the primary sensing vector to the alternate sensing vector for sensing further electrical cardiac signals for use in warnings are eventually routed to an external device programmer operated under the control of a clinician, at step 504, for use during an in-clinic consultation with the patient. Alternatively, at step 504, the clinician can simply initiate a “full manual” assessment, even in the absence of any detected drop in signal amplitude.

During the in-clinic consultation, the programmer systematically tests all possible sensing configurations, at step 506, under the control of the clinician by sending control signals to the pacer/ICD to test or map various sensing vectors. At step 508, in response to commands from the programmer, the pacer/ICD senses cardiac signals within the patient using various sensing vectors and then transmits the sensed signals to the device programmer for clinician review. These may be the same tests described above in connection with step 206 of FIG. 3, but performed under the control of the programmer while under clinician supervision. The programmer receives and analyzes the cardiac signals (also at step 506) to assess sensing efficacy and to identify a set of candidate vectors for clinician review.

The procedure of steps 504 and 506 continues until all sensing configurations to be tested have been tested and the results compiled. Depending upon the number of electrodes on the various leads there might be a fairly large number of sensing vectors to be tested. If so, various techniques may be exploited for reducing the total number of sensing tests to be performed. See, e.g., U.S. patent application Ser. No. 12/703,069, filed Feb. 9, 2010, of Rosenberg et al., entitled “Systems and Methods for Optimizing Multi-Site Cardiac Pacing and Sensing Configurations for use with an Implantable Medical Device.”

At step 510, the device programmer generates a report listing candidate sensing vectors and providing sample signals and various sensing efficacy parameters (such as values representative of noise levels, signal amplitudes, etc.) In one example, the following table of results is presented (wherein the signal amplitude values are R-wave amplitudes as measured by the implanted device):

controlling further therapy, where improved sensing is defined in terms of improved signal amplitude, slew rate, bipolar spacing, interelectrode distance or clinician-specified criteria, or in terms of the shortest interelectrode distance that can be selected so long as amplitude and slew rate are adequate for sensing.

FIG. 11 provides some further details. In the figure, the processing of the primary sensing channel is shown on the left side of the drawing; the background processing of the alternate sensing channel is shown on the right side of the drawing. Beginning at step 900, the pacer/ICD senses IEGM signals using a primary vector connected to the primary sensing channel, such as by sensing via LVtip—LVring1. At step 902, the pacer/ICD controls device therapy based on the cardiac signals sensed on the primary sensing channel. Concurrently, beginning at step 904, the pacer/ICD also periodically or continuously tests all other available sensing vectors by systematically connecting various sensing vectors to the alternate channel to assess sensing efficacy to identify sensing vectors (if any) that are more effective than the current primary vector. If such a vector is identified at step 906, the processing proceeds to step 908 wherein the pacer/ICD connects the newly identified alternate vector to the primary sensing channel as a new primary sensing vector. At step 910, a silent alarm is generated to the clinician and suitable diagnostics are generated. Such diagnostics set forth the reason for the switch to the alternate sensing vector, including exemplary IEGM signals obtained using the previous primary vector and IEGM signals obtained using the new primary vector.

Thus, with reference to FIGS. 1-11, a variety of exemplary sensing vector configuration adjustment or optimization systems and techniques have been described. The systems and techniques can be used, where appropriate, in conjunction with other optimization procedures. See, for example, the QuickStim and QuickSense techniques of the Rosenberg et al. patent application, cited above, as well as various QuickOpt™ techniques. QuickOpt™ techniques (or other techniques for setting AV/PV/VV delays) are discussed in the following patents and patent applications: U.S. patent application Ser. No. 10/703,070, filed Nov. 5, 2003, entitled “Methods for Ventricular Pacing” (Attorney Docket No. A03P1074), now abandoned; U.S. patent application Ser. No. 10/974,123, filed Oct. 26, 2004 (Attorney Docket No. A03P1074US01), now abandoned; U.S. patent application Ser. No. 10/986,273, filed Nov. 10, 2004 (Attorney Docket No. A03P1074US02), now U.S. Pat. No. 7,590,446; U.S. patent application Ser. No. 10/980,140, filed Nov. 1, 2004 (Attorney Docket No. A03P1074US03), now abandoned; U.S. patent application Ser. No. 11/129,540, filed May 13, 2005 (Attorney Docket No. A03P1074US04), now abandoned; U.S. patent application Ser. No. 11/952,743, filed Dec. 7, 2007 (Attorney Docket No. A07P1179). See, also, U.S. patent application Ser. No. 12/328,605, filed Dec. 4, 2008, entitled “Systems and Methods for Controlling Ventricular Pacing in Patients with Long Intra-Atrial Conduction Delays” (Attorney Docket No. A08P1067); and U.S. patent application Ser. No. 12/132,563, filed Jun. 3, 2008, entitled “Systems and Methods for determining Intra-Atrial Conduction Delays using Multi-Pole Left Ventricular Pacing/Sensing Leads” (Attorney Docket No. A08P1021), now U.S. Pub. App. 2009/0299423A1. See, further, U.S. Pat. No. 7,248,925, to Bruhns et al., entitled “System and Method for Determining Optimal Atrioventricular Delay based on Intrinsic Conduction Delays.”

Note that QuickStim and QuickSense may be regarded as trademarks. See, also, the various techniques described in U.S. patent application Ser. No. 11/750,153, filed May 17, 2007, entitled “Expedited Set-Up of Multi-Electrode Lead (MEL)” (Attorney Docket No. A07P3020).

Note also that in the examples described herein the multi-pole ventricular lead is an LV lead, but it should be understood that aspects of the invention are applicable to multi-pole RV leads. Indeed, at least some of the techniques described herein are also generally applicable to implementations wherein both the LV and RV have multi-pole leads. Still further, the techniques are applicable to multi-pole atrial leads, implanted on or in either the RA or the LA. As such, at least some of the techniques described herein are generally applicable to adjusting, switching or optimizing sensing configurations as applied to leads implanted on or in any of the four chambers of the heart. The techniques are also applicable to non-multi-pole leads, such as standard bipolar leads where there are multiple bipolar leads that have been implanted.

It should also be understood that any optimal sensing vectors mentioned herein are not necessarily absolutely optimal in any quantifiable or mathematical sense. As can be appreciated, what constitutes an “optimal” sensing vector depends on the criteria used for judging the resulting sensing performance, which can be subjective in the minds of some clinicians. The sensing configurations determined by the techniques described herein represent, at least, “preferred” sensing configurations. Clinicians may choose to adjust or alter the selection via device programming for particular patients, at their discretion.

Still further, although primarily described with respect to examples having a pacer/ICD, other implantable medical devices may be equipped to exploit the techniques described herein such as CRT devices and CRT-D devices (i.e. a CRT device also equipped to deliver defibrillation shocks and antitachycardia pacing therapy for ventricular tachyarrhythmias in addition to biventricular pacing) or CRT-P devices (i.e. a CRT device equipped to deliver biventricular pacing) or any appropriate implantable “pulse generator” device, which can include devices that sense various non-cardiac electrical signals in other tissues or organs besides the heart. For the sake of completeness, an exemplary pacer/ICD will now be described that provides CRT and that includes components for performing at least some of the functions and steps already described.

### Exemplary Pacer/ICD

With reference to FIGS. 12 and 13, a description of an exemplary pacer/ICD will now be provided. FIG. 12 provides a simplified block diagram of the pacer/ICD, which is a dual-chamber stimulation device capable of treating both fast and slow arrhythmias with stimulation therapy, including cardioversion, defibrillation, and pacing stimulation, and also capable of delivering CRT. To provide other atrial chamber pacing stimulation and sensing, pacer/ICD 10 is shown in electrical communication with a heart 1012 by way of a left atrial lead 1020 having an atrial tip electrode 1022 and an atrial ring electrode 1023 implanted in the atrial appendage. Pacer/ICD 10 is also in electrical communication with the heart by way of a right ventricular lead 1030 having, in this embodiment, a ventricular tip electrode 1032, a right ventricular ring electrode 1034, a right ventricular (RV) coil electrode 1036, and a superior vena cava (SVC) coil electrode 1038. Typically, the right ventricular lead 1030 is transvenously inserted into the heart so as to place the RV coil electrode 1036 in the right ventricular apex, and the SVC coil electrode 1038 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a multi-pole LV lead 1024 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. An LV lead may also be an epicardial lead placed via a thoracotomy on the surface of the heart and or endocardial LV placement. Accordingly, an exemplary LV lead 1024 connected to an appropriate pulse generator is designed to sense ventricular cardiac signals and to deliver left ventricular pacing therapy using a set of four left ventricular electrodes 1026₁, 1026₂, 1026₃, and 1026₄ (thereby providing a Quadrapole lead), left atrial pacing therapy using at least a left atrial ring electrode 1027, and shocking therapy using at least a left atrial coil electrode 1028. The 1026₁ LV electrode may also be referred to as a “tip” or “distal” LV electrode. The 1026₄ LV electrode may also be referred to as a “proximal” LV electrode. By proximal, it is meant closer to the terminal pin of the lead. In other examples, more or fewer LV electrodes are provided. Although only three leads are shown in FIG. 12, it should also be understood that additional leads (with one or more pacing, sensing and/or shocking electrodes) might be used and/or additional electrodes might be provided on the leads already shown, such as additional electrodes on the RV lead.

A simplified block diagram of internal components of pacer/ICD 10 is shown in FIG. 13. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 1040 for pacer/ICD 10, shown schematically in FIG. 13, is often referred to as the “can”, “case” or “case electrode” and may be programmably selected to act as the return electrode for all “unipolar” modes. The housing 1040 may further be used as a return electrode alone or in combination with one or more of the coil electrodes, 1028, 1036 and 1038, for shocking purposes. The housing 1040 further includes a connector (not shown) having a plurality of terminals, 1042, 1043, 1044₁-1044₄, 1046, 1048, 1052, 1054, 1056 and 1058 (shown schematically and, for convenience, the names of the electrodes to which they are connected are shown next to the terminals). As such, to achieve right atrial sensing and pacing, the connector includes at least a right atrial tip terminal (AR TIP) 1042 adapted for connection to the atrial tip electrode 1022 and a right atrial ring (AR RING) electrode 1043 adapted for connection to right atrial ring electrode 1023. To achieve left chamber sensing, pacing and shocking, the connector includes a left ventricular tip terminal (VL₁ TIP) 1044₁ and additional LV electrode terminals 1044₂-1044₄ for the other LV electrodes of the Quadrapole LV lead.

The connector also includes a left atrial ring terminal (AL RING) 1046 and a left atrial shocking terminal (AL COIL) 1048, which are adapted for connection to the left atrial ring electrode 1027 and the left atrial coil electrode 1028, respectively. To support right chamber sensing, pacing and shocking, the connector further includes a right ventricular tip terminal (VR TIP) 1052, a right ventricular ring terminal (VR RING) 1054, a right ventricular shocking terminal (VR COIL) 1056, and an SVC shocking terminal (SVC COIL) 1058, which are adapted for connection to the right ventricular tip electrode 1032, right ventricular ring electrode 1034, the VR coil electrode 1036, and the SVC coil electrode 1038, respectively.

At the core of pacer/ICD 10 is a programmable microcontroller 1060, which controls the various modes of stimulation therapy. As is well known in the art, the microcontroller 1060 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 1060 includes the ability to process or monitor input signals (data) as controlled by a program code stored in a designated block of memory. The details of the design and operation of the microcontroller 1060 are not critical to the invention. Rather, any suitable microcontroller 1060 may be used that carries out the functions described herein. The use of microprocessor-based control circuits for performing timing and data analysis functions are well known in the art.

As shown in FIG. 13, an atrial pulse generator 1070 and a ventricular pulse generator 1072 generate pacing stimulation pulses for delivery by the right atrial lead 1020, the right ventricular lead 1030, and/or the LV lead 1024 via an electrode configuration switch 1074. It is understood that in order to provide stimulation therapy in each of the four chambers of the heart, the atrial and ventricular pulse generators, 1070 and 1072, may include dedicated, independent pulse generators, multiplexed pulse generators or shared pulse generators. The pulse generators, 1070 and 1072, are controlled by the microcontroller 1060 via appropriate control signals, 1076 and 1078, respectively, to trigger or inhibit the stimulation pulses.

The microcontroller 1060 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 1074 includes a plurality of switches for connecting the desired electrodes to the appropriate I/O circuits, thereby providing complete electrode programmability. Accordingly, the switch 1074, in response to a control signal 1080 from the microcontroller 1060, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 1082 and ventricular sensing circuits 1084 may also be selectively coupled to the right atrial lead 1020, LV lead 1024, and the right ventricular lead 1030, through the switch 1074 for detecting the presence of cardiac activity in each of the four chambers of the heart. Accordingly, the atrial (ATR. SENSE) and ventricular (VTR. SENSE) sensing circuits, 1082 and 1084, may include dedicated sense amplifiers, multiplexed amplifiers or shared amplifiers. These sensing amplifiers provide various sensing channels. The switch 1074 determines the “sensing polarity” of the cardiac signal by selectively closing the appropriate switches, as is also known in the art. In this way, the clinician may program the sensing polarity independent of the stimulation polarity. Each sensing circuit, 1082 and 1084, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 1082 and 1084, are connected to the microcontroller 1060 which, in turn, are able to trigger or inhibit the atrial and ventricular pulse generators, 1070 and 1072, respectively, in a demand fashion in response to the absence or presence of cardiac activity in the appropriate chambers of the heart.

For arrhythmia detection, pacer/ICD 10 utilizes the atrial and ventricular sensing circuits, 1082 and 1084, to sense cardiac signals to determine whether a rhythm is physiologic or pathologic. As used in this section “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 1060 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 1090. The data acquisition system 1090 is configured to acquire intracardiac electrogram signals, convert the raw analog data into a digital signal, and store the digital signals for later processing and/or telemetric transmission to an external device 1102. The data acquisition system 1090 is coupled to the right atrial lead 1020, the LV lead 1024, and the right ventricular lead 1030 through the switch 1074 to sample cardiac signals across any pair of desired electrodes. The microcontroller 1060 is further coupled to a memory 1094 by a suitable data/address bus 1096, wherein the programmable operating parameters used by the microcontroller 1060 are stored and modified, as required, in order to customize the operation of pacer/ICD 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 1094 through a telemetry circuit 1100 in telemetric communication with the external device 1102, such as a programmer, transtelephonic transceiver or a diagnostic system analyzer. The telemetry circuit 1100 is activated by the microcontroller by a control signal 1106. The telemetry circuit 1100 advantageously allows intracardiac electrograms and status information relating to the operation of pacer/ICD 10 (as contained in the microcontroller 1060 or memory 1094) to be sent to the external device 1102 through an established communication link 1104. Pacer/ICD 10 further includes an accelerometer or other physiologic sensor 1108, commonly referred to as a “rate-responsive” sensor because it is typically used to adjust pacing stimulation rate according to the exercise state of the patient. However, the physiological sensor 1108 may further be used to detect changes in cardiac output, changes in the physiological condition of the heart, or diurnal changes in activity (e.g., detecting sleep and wake states) and to detect arousal from sleep. Accordingly, the microcontroller 1060 responds by adjusting the various pacing parameters (such as rate, AV delay, VV delay, etc.) at which the atrial and ventricular pulse generators, 1070 and 1072, generate stimulation pulses. While shown as being included within pacer/ICD 10, it is to be understood that the physiologic sensor 1108 may also be external to pacer/ICD 10, yet still be implanted within or carried by the patient. A common type of rate responsive sensor is an activity sensor incorporating an accelerometer or a piezoelectric crystal, which is mounted within the housing 1040 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 1110, which provides operating power to all of the circuits shown in FIG. 13. The battery 1110 may vary depending on the capabilities of pacer/ICD 10. If the system only provides low voltage therapy, a lithium iodine or lithium copper fluoride cell typically may be utilized. For pacer/ICD 10, which employs shocking therapy, the battery 1110 should be capable of operating at low current drains for long periods, and then be capable of providing high-current pulses (for capacitor charging) when the patient requires a shock pulse. The battery 1110 should also have a predictable discharge characteristic so that elective replacement time can be detected. Accordingly, appropriate batteries are employed.

As further shown in FIG. 13, pacer/ICD 10 is shown as having an impedance measuring circuit 1112, which is enabled by the microcontroller 1060 via a control signal 1114. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; detecting the opening of heart valves, and measuring values useful for assessing current drain and device longevity, etc. Impedance can also be used to detect a complete lead failure. (A high level of noise can also indicate lead failure.) In response, the clinician and/or patient is alerted and inappropriate therapies are withheld. Additionally, the device might be equipped to automatically switch to integrated bipolar for pacing and sensing if the system determines that only the RVring electrode is compromised. The impedance measuring circuit 1112 is advantageously coupled to the switch 1174 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 1060 further controls a shocking circuit 1116 by way of a control signal 1118. The shocking circuit 1116 generates shocking pulses of low (up to 0.5 joules), moderate (0.5-10 joules) or high energy (11 to 40 joules or more), as controlled by the microcontroller 1060. Such shocking pulses are applied to the heart of the patient through at least two shocking electrodes, and as shown in this embodiment, selected from the left atrial coil electrode 1028, the RV coil electrode 1036, and/or the SVC coil electrode 1038. The housing 1040 may act as an active electrode in combination with the RV electrode 1036, or as part of a split electrical vector using the SVC coil electrode 1038 or the left atrial coil electrode 1028 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 7-40 joules), delivered asynchronously (since R-waves may be too disorganized), and pertaining exclusively to the treatment of fibrillation. Accordingly, the microcontroller 1060 is capable of controlling the synchronous or asynchronous delivery of the shocking pulses.

An internal warning device 1099 may be provided for generating perceptible warning signals to the patient via vibration, voltage or other methods.

Insofar as sensing vector reprogramming is concerned, the microcontroller includes an on-board sensing configuration controller 1101 operative to perform or control the pacer/ICD-based techniques described above. The on-board controller includes a primary vector sensing system 1115 operative to sense electrical signals within the patient using a primary sensing vector connected to a primary sensing channel for use in controlling the delivery of therapy and an alternate vector sensing system 1117 operative to sense electrical signals within the patient using an alternate sensing vector connected to an alternate sensing channel. The controller also includes a sensing issue detection system 1103 operative to detect a sensing issue affecting a current or primary sensing vector based on electrical cardiac signals sensed via the various sense amplifiers along various sensing channels, specifically a significant drop in peak signal amplitude indicative of a potential sensing problem. The on-board controller also includes a sensing configuration assessment system 1105 operative to assess whether one or more alternate sensing vectors provide improved cardiac signal sensing over the primary sensing vector, using the various criteria discussed above. A sensing configuration switching system 1107 is operative to switch the primary sensing channel from the primary sensing vector to the alternate sensing vector for sensing further cardiac signals for use in controlling further therapy (assuming this feature has been enabled in advance by the clinician.) A background sensing configuration analysis system 1109 may be provided to control or perform the background sensing vector analysis techniques of FIGS. 10 and 11. The aforementioned monitoring mode, where the device monitors for a drop in signal amplitude and assesses whether an alternate sensing vector provides improved cardiac signal sensing but does not make an changes in the sensing configuration, is controlled by monitoring mode controller 1111. CRT is controlled by a CRT controller 1113.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

As noted, at least some of the techniques described herein can be performed by (or under the control of) an external device. For the sake of completeness, a detailed description of an exemplary device programmer will now be provided.

### Exemplary External Programmer

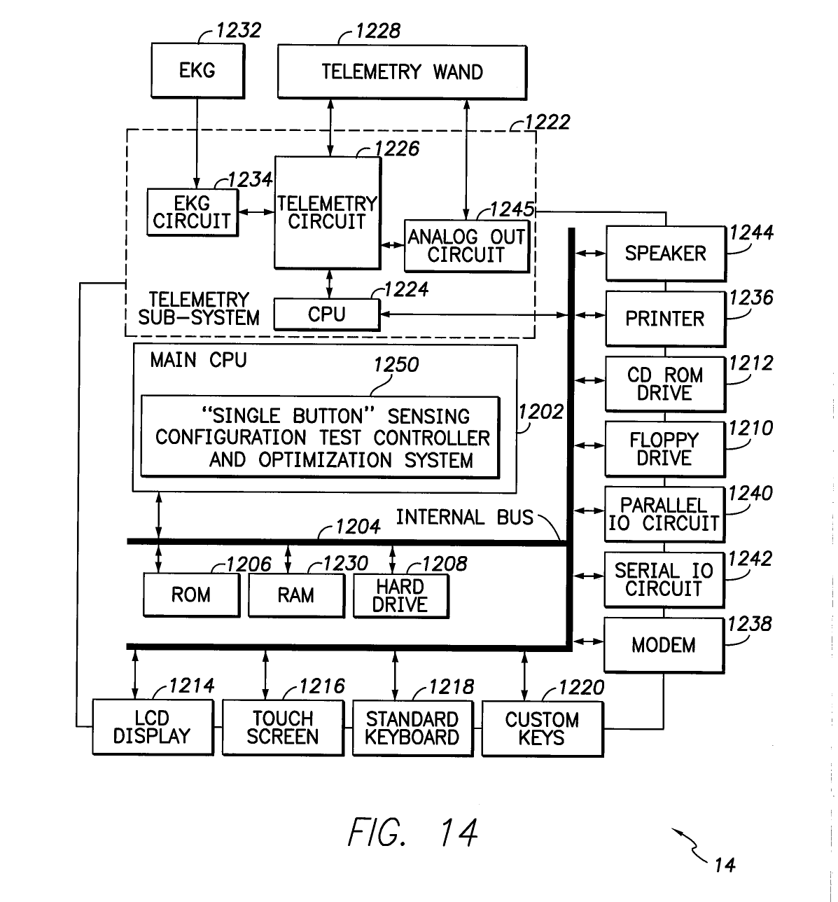

FIG. 14 illustrates pertinent components of an external programmer 14 for use in programming the pacer/ICD of FIGS. 12 and 13 and for performing the above-described optimization techniques. For the sake of completeness, other device programming functions are also described herein. Generally, the programmer permits a clinician or other user to program the operation of the implanted device and to retrieve and display information received from the implanted device such as IEGM data and device diagnostic data. Additionally, the external programmer can be optionally equipped to receive and display electrocardiogram (EKG) data from separate external EKG leads that may be attached to the patient. Depending upon the specific programming of the external programmer, programmer 14 may also be capable of processing and analyzing data received from the implanted device and from the EKG leads to, for example, render preliminary diagnosis as to medical conditions of the patient or to the operations of the implanted device.

Now, considering the components of programmer 14, operations of the programmer are controlled by a CPU 1202, which may be a generally programmable microprocessor or microcontroller or may be a dedicated processing device such as an application specific integrated circuit (ASIC) or the like. Software instructions to be performed by the CPU are accessed via an internal bus 1204 from a read only memory (ROM) 1206 and random access memory 1230. Additional software may be accessed from a hard drive 1208, floppy drive 1210, and CD ROM drive 1212, or other suitable permanent mass storage device. Depending upon the specific implementation, a basic input output system (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 1214 or other suitable computer display device. To this end, the CPU may, for example, display a menu of specific programmable parameters of the implanted device to be programmed or may display a menu of types of diagnostic data to be retrieved and displayed. In response thereto, the physician enters various commands via either a touch screen 1216 overlaid on the LCD display or through a standard keyboard 1218 supplemented by additional custom keys 1220, such as an emergency WI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

Once all pacing leads are mounted and the pacing device is implanted, the various parameters are programmed. Typically, the physician initially controls the programmer 14 to retrieve data stored within any implanted devices and to also retrieve EKG data from EKG leads, if any, coupled to the patient. To this end, CPU 1202 transmits appropriate signals to a telemetry subsystem 1222, which provides components for directly interfacing with the implanted devices, and the EKG leads. Telemetry subsystem 1222 includes its own separate CPU 1224 for coordinating the operations of the telemetry subsystem. Main CPU 1202 of programmer communicates with telemetry subsystem CPU 1224 via internal bus 1204. Telemetry subsystem additionally includes a telemetry circuit 1226 connected to telemetry wand 1228, which, in turn, receives and transmits signals electromagnetically from a telemetry unit of the implanted device. The telemetry wand is placed over the chest of the patient near the implanted device to permit reliable transmission of data between the telemetry wand and the implanted device. Herein, the telemetry subsystem is shown as also including an EKG circuit 1234 for receiving surface EKG signals from a surface EKG system 1232. In other implementations, the EKG circuit is not regarded as a portion of the telemetry subsystem but is regarded as a separate component.

Typically, at the beginning of the programming session, the external programming device controls the implanted devices via appropriate signals generated by the telemetry wand to output all previously recorded patient and device diagnostic information. Patient diagnostic information includes, for example, recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like. Data retrieved from the pacer/ICD also includes the data stored within the recalibration database of the pacer/ICD (assuming the pacer/ICD is equipped to store that data.) Data retrieved from the implanted devices is stored by external programmer 14 either within a random access memory (RAM) 1230, hard drive 1208 or within a floppy diskette placed within floppy drive 1210. Additionally, or in the alternative, data may be permanently or semi-permanently stored within a compact disk (CD) or other digital media disk, if the overall system is configured with a drive for recording data onto digital media disks, such as a write once read many (WORM) drive.

Once all patient and device diagnostic data previously stored within the implanted devices is transferred to programmer 14, the implanted devices may be further controlled to transmit additional data in real time as it is detected by the implanted devices, such as additional IEGM data, lead impedance data, and the like. Additionally, or in the alternative, telemetry subsystem 1222 receives EKG signals from EKG leads 1232 via an EKG processing circuit 1234. As with data retrieved from the implanted device itself, signals received from the EKG leads are stored within one or more of the storage devices of the external programmer. Typically, EKG leads output analog electrical signals representative of the EKG. Accordingly, EKG circuit 1234 includes analog to digital conversion circuitry for converting the signals to digital data appropriate for further processing within the programmer. Depending upon the implementation, the EKG circuit may be configured to convert the analog signals into event record data for ease of processing along with the event record data retrieved from the implanted device. Typically, signals received from the EKG leads are received and processed in real time.

Thus, the programmer receives data both from the implanted devices and from optional external EKG leads. Data retrieved from the implanted devices includes parameters representative of the current programming state of the implanted devices. Under the control of the physician, the external programmer displays the current programmable parameters and permits the physician to reprogram the parameters. To this end, the physician enters appropriate commands via any of the aforementioned input devices and, under control of CPU 1202, the programming commands are converted to specific programmable parameters for transmission to the implanted devices via telemetry wand 1228 to thereby reprogram the implanted devices. Prior to reprogramming specific parameters, the physician may control the external programmer to display any or all of the data retrieved from the implanted devices or from the EKG leads, including displays of EKGs, IEGMs, and statistical patient information. Any or all of the information displayed by programmer may also be printed using a printer 1236.

Programmer/monitor 14 also includes a modem 1238 to permit direct transmission of data to other programmers via the public switched telephone network (PSTN) or other interconnection line, such as a T1 line or fiber optic cable. Depending upon the implementation, the modem may be connected directly to internal bus 1204 may be connected to the internal bus via either a parallel port 1240 or a serial port 1242. Other peripheral devices may be connected to the external programmer via parallel port 1240 or a serial port 1242 as well. Although one of each is shown, a plurality of input output (10) ports might be provided. A speaker 1244 is included for providing audible tones to the user, such as a warning beep in the event improper input is provided by the physician. Telemetry subsystem 1222 additionally includes an analog output circuit 1245 for controlling the transmission of analog output signals, such as IEGM signals output to an EKG machine or chart recorder.

Insofar as sensing vector reprogramming is concerned, the main CPU may include a “single button” sensing configuration test controller and optimization system 1250 operative to perform or control the programmer-based techniques described above. To this end, controller 1250 may include various sub-components, not separately shown in the figure, such as components operative to: receive signals indicating the detection of a sensing issue within a pacer/ICD affecting a current or primary sensing vector; assess whether an alternate sensing vector of the pacer/ICD provides improved cardiac signal sensing over a current primary sensing vector; and generate programming commands to switch the primary sensing channel of the pacer/ICD from the primary sensing vector to the alternate sensing vector for sensing further cardiac signals for use in controlling further therapy. Controller 1250 also controls the generation of the various displays and reports discussed above, as well as to receive clinician input and instructions.

Depending upon the implementation, the various components of the CPU may be implemented as separate software modules or the modules may be combined to permit a single module to perform multiple functions. In addition, although shown as being components of the CPU, some or all of these components may be implemented separately, using ASICs or the like.

With the programmer configured as shown, a clinician or other user operating the external programmer is capable of retrieving, processing and displaying a wide range of information received from the implanted device and to reprogram the implanted device if needed.

The descriptions provided herein with respect to FIG. 13 are intended merely to provide an overview of the operation of programmer and are not intended to describe in detail every feature of the hardware and software of the programmer and is not intended to provide an exhaustive list of the functions performed by the programmer.

In general, while the invention has been described with reference to particular embodiments, modifications can be made thereto without departing from the scope of the invention. Note also that the term “including” as used herein is intended to be inclusive, i.e. “including but not limited to.”

FIG. 15 is an illustrative example from two dual chamber pacemakers, each with bipolar leads in the atrium and the ventricle showing different configurations for the electrogram and hence sensing circuits. Selecting one pair over another may be based on one or more factors including but not limited to signal amplitude, either of the rectified signal or peak to peak, the slew rate of the signal or the bipole separation as long as the amplitude is sufficient for sensing purposes.

What is claimed is:

1. A method for use with an implantable medical device capable of sensing electrical signals along a plurality of sensing channels connected to a selectable sensing vector, the method comprising:
sensing electrical signals within the patient using a primary sensing vector connected to a primary sensing channel for use in controlling the delivery of therapy;
sensing additional electrical signals within the patient using an alternate sensing vector connected to an alternate sensing channel; and
while continuing to sense signals using the primary sensing vector, assessing whether the alternate sensing vector provides improved sensing over the primary sensing vector and, if so, switching the primary sensing channel from the primary sensing vector to the alternate sensing vector for sensing further electrical signals for use in controlling further therapy.

2. The method of claim 1 including a preliminary step of detecting a significant change in a selected parameter of the electrical signals sensed along the primary sensing vector and wherein the steps of sensing additional electrical signals within the patient using an alternate sensing vector and assessing whether the alternate sensing vector provides improved sensing are performed in response to the detection of the significant change in the selected parameter.

3. The method of claim 2 wherein the selected parameter includes one or more of a trend in signal amplitude, a trend in slew rate, or a trend in other clinician-specified criteria.

4. The method of claim 2 wherein the steps are all performed by the implantable medical device.

5. The method of claim 2 wherein an external system is used in conjunction with the implantable medical device and wherein at least some of the steps are performed by the external system.

6. The method of claim 5 wherein the external system sends commands to the implantable device to initiate the assessment of whether the alternate sensing vector provides improved signal sensing over the primary sensing vector.

7. The method of claim 5 wherein commands sent to the implantable device are generated under the supervision of a clinician.

8. The method of claim 2 wherein the electrical signals are electrical cardiac signals.

9. The method of claim 8 wherein assessing whether an alternate sensing vector provides improved sensing includes:
systematically sensing cardiac signals within the patient using each of a plurality of candidate sensing vectors;
assessing a degree of sensing efficacy for each of the candidate vectors; and
identifying candidate vectors that offer greater sensing efficacy than the initial primary sensing vector.

10. The method of claim 2 wherein the steps of detecting a significant change in electrical signals and assessing whether an alternate sensing vector provides improved signal sensing are performed periodically.

11. The method of claim 2 wherein the steps of detecting a significant change in the electrical signals and assessing whether an alternate sensing vector provides improved signal sensing are performed to automatically change the sensing configuration.

12. The method of claim 2 wherein the steps of detecting a significant change in the electrical signals and assessing whether an alternate sensing vector provides improved signal sensing are performed in a monitor mode where changes are only made to the sensing configuration pending clinician review.

13. The method of claim 2 wherein the step of detecting a significant change in the electrical signals is performed relative to a nominal sensing amplitude.

14. The method of claim 1 wherein assessing whether an alternate sensing vector provides improved sensing is performed by the device.

15. The method of claim 1 wherein assessing whether an alternate sensing vector provides improved sensing includes generating a report for transmission to an external system identifying candidate vectors.

16. The method of claim 1 wherein assessing whether an alternate sensing vector provides improved sensing is performed under the control of the external system based on signals received from the implantable device.

17. A system for use with an implantable medical device capable of sensing electrical signals along a plurality of sensing channels connected to a selectable sensing vector, the system comprising:
a primary signal sensing system operative to sense electrical signals within the patient using a primary sensing vector connected to a primary sensing channel for use in controlling the delivery of therapy;
an alternate signal sensing system operative to sense electrical signals within the patient using an alternate sensing vector connected to an alternate sensing channel;
an sensing configuration assessment system operative, while the primary signal sensing system continues to sense electrical signals, to assess whether the alternate sensing vector provides improved signal sensing over the primary sensing vector; and
a sensing configuration switching system operative to selectively switch the primary sensing channel from the primary sensing vector to the alternate sensing vector for sensing further signals for use in controlling further therapy.

18. The system of claim 17 further including a signal amplitude change detection system operative to detect a significant change in a selected parameter of the electrical signals sensed along the primary sensing vector.

19. A system for use with an implantable medical device capable of sensing electrical signals along a plurality of sensing channels connected to a selectable sensing vector, the system comprising:
means for sensing electrical signals within the patient using a primary sensing vector connected to a primary sensing channel for use in controlling the delivery of therapy;
means for sensing additional electrical signals within the patient using an alternate sensing vector connected to an alternate sensing channel; and
means, operative while the means for sensing electrical signals continues to sense signals using the primary sensing vector, for assessing whether the alternate sensing vector provides improved sensing over the primary sensing vector and, in response, for switching the primary sensing channel from the primary sensing vector to the alternate sensing vector for sensing further electrical signals for use in controlling further therapy.

20. A method for use with an implantable medical device capable of sensing electrical signals along a plurality of sensing channels connected to a selectable set of sensing vectors, the method comprising:
sensing electrical signals within the patient using a primary sensing vector connected to a primary sensing channel for use in controlling the delivery of therapy;
detecting a significant change in a slew rate of the electrical signals sensed along the primary sensing vector; and
assessing whether an alternate sensing vector provides improved signal sensing over the primary sensing vector and, if so, selectively switching the primary sensing channel from the primary sensing vector to the alternate sensing vector for sensing further electrical signals for use in controlling further therapy.
